(19) United States
(12) Patent Application Publication
(10) Pub. No.: US 2012/0251579 A1
(43) Pub. Date: Oct. 4, 2012

(54) COMPOSITIONS FOR GENERATING AN ANTIGEN SPECIFIC IMMUNE RESPONSE

(75) Inventor: Lars Zender, Hannover (DE)

(73) Assignee: Helmholtz-Zentrum Fuer Infektionsforschung GmbH, Braunschweig (DE)

(21) Appl. No.: 13/386,590

(22) PCT Filed: Jul. 30, 2010
(86) PCT No.: PCT/EP2010/061161

§ 371 Date: Apr. 9, 2012

(30) Foreign Application Priority Data
Jul. 30, 2009 (EP) 09166911.9

(51) Int. Cl. A61K 35/12; C12N 5/071; C12N 5/10; C12P 21/02; C07F 9/00; A61K 39/00; A61K 31/496; C07D 403/04; A61K 31/555; A61P 35/00; C12N 15/85

(52) U.S. Cl. 424/277.1; 424/93.7; 435/354; 435/375; 435/69.6; 435/455; 514/254.05; 544/370; 514/188; 546/5

(57) ABSTRACT

The present invention provides for the purposeful utilisation of the induction of senescence in eukaryotic cells for induction of an antigen specific immune response. Such cells can be normal cells, pre-malignant and malignant cells as well as virally or bacterially infected cells, for the generation of an immune response, preferably a cellular or humoral immune response comprising T-cells and/or B-cells, whose immune response is directed specifically against antigens from those cells in which senescence was induced and then comprises an immune response against the senescent cells itself as well as to the non-senescent counterparts harbouring the same antigens.

The present invention relates to the purposeful utilisation of the induction of senescence in eukaryotic cells for induction of an immune response. Such cells can be normal cells, pre-malignant and malignant cells as well as virally or bacterially infected cells, for the generation of an immune response, preferably a cellular or humoral immune response comprising T-cells and/or B-cells, whose immune response is directed specifically against antigens from those cells in which senescence was induced and then comprises an immune response against the senescent cells itself as well as to the non-senescent counterparts harbouring the same antigens. The immune response induced by presence of senescent malignant cells is directed against malignant cells, both in their senescent state and in their non-senescent state, e.g. in a normal cell cycle phase, including a phase of proliferation of malignant cells, and including pre-malignant cells, e.g. chronically virally or bacterially infected cells, e.g. chronically infected liver cells. For utilisation of the senescence induced antigen specific immune response, specifically directed against malignant cells, the invention provides pharmaceutical compositions, e.g. for use as a medicament, especially for use in the treatment of malignant cells, and the use of compounds for the production of pharmaceutical compositions, which compounds generate senescent premalignant or malignant cells, which are the causative agent of the generation of the malignant cell—specific immune response, and further provides pharmaceutical compositions comprising the senescent malignant cells as the causative agent for generating the immune response directed against the malignant cells, as well as pharmaceutical compositions comprising the immune cells, especially T-cells and B-cells, which are specific for the malignant cells, which immune cells are generated by contacting non-primed immune cells with senescent malignant cells in the presence and/or in the absence of antigen presenting cells (APCs). Further, the invention relates to the utilisation of the induction of a specific (antigen specific) immune response effected by the presence of senescent cells for the production of antigen—specific immune cells in an experimental animal, i.e. in a non-human mammal, e.g. in a mouse. For the purposes of the invention, the term “medicament” is used interchangeably with the term “pharmaceutical composition” or “pharmaceutical agent”.

In contrast to the state of art which shows that the artificial induction of senescence raises an immune response by the innate immune system, i.e. a non-adaptive immune response, the present invention is based on the finding that the cellular senescence program is a very efficient inducer of an adaptive immune response, e.g. T-cells and B-cells are induced. Due to their naturally occurring secretory phenotype and an increased expression of cellular adhesion molecules, senescent cells are an ideal target for phagocytosis by APC, which then cross-present antigens from the senescent cells (in the case of the experimental data presented here antigens are peptides from the Kras oncogene) to effectively activate B- or T-Cells. Some cell types (e.g. liver cells), upon senescence induction, will also be able to directly present antigens to adaptive immune cells. The specificity of the cells and of antibody secreted by respective specific B-cells is directed against antigen contained in the senescent malignant cell, e.g. presented to the immune system by MHC I and/or MHC II of the senescent malignant cell, so that the specificity of the immune response is e.g. specific for the intracellular antigen e.g. in a malignant or in a virally infected cell.

Accordingly, in certain embodiments of the invention, the generation of specific T-cells and/or B-cells by contacting immune cells with senescent malignant cells or non malignant senescent cells overexpressing a certain antigen, e.g. a tumour antigen, is described, which can be used for the production of antibodies, the antibody having specificity for an antigen that is expressed by the malignant cell, e.g. after transformation of the malignant cell due to transfection or transformation with an antigen encoding nucleic acid sequence. In this embodiment, the cell expressing the antigen is considered a malignant cell for the purposes of this invention, and the cell can be selected essentially from any type of cell. The advantage of using the stimulation of immune cells with senescent malignant cells or non malignant senescent cells overexpressing a certain tumour antigen, which are cells expressing an antigen, for generating a T-cell and/or a B-cell secreting an antibody specific for the antigen is the high specificity and high effectiveness of the generation of a specific immune response by contacting immune cells, preferably non-primed immune cells, with senescent malignant cells. Preferably, the malignant cell in the embodiments of the invention is a cell, present in vitro or in vivo in a human patient or in an experimental animal, is expressing the antigen against which an immune response is desired, which malignant cell is present in its state of senescence and in contact with the immune cells, either in vitro or in vivo, e.g. in a human patient or in an experimental animal, i.e. in a non-human mammal. Preferably, the induction of an antigen-specific immune response is monitored, e.g. by using an ELISPOT assay to detect antigen-specific T-Lymphocytes or by using a B-cell antibody ELISA.

### State of the art

WO 2009/042798 describes a treatment of fibrosis of the liver by administration of an agent promoting the senescence of myofibroblasts in fibrotic tissue, optionally in combination with the administration of an immuno stimulant for activating or recruiting the innate immune system in fibrotic tissue. For inducing the senescence of myofibroblasts, and a viral expression vector encoding p53, p21/Cip1/Waf1 cyclin dependent kinase inhibitor or a miR-34 class of microRNA, or encoding a short-hairpin RNA molecule for causing post-transcriptional silencing of cycline-dependant kinases 2 or 4 is used.

Xue et al in Nature, 656-660 (2007) describe that innate immune cells were activated in athymic nude mice against intraspleenically injected purified embryonic liver progenitor cells, that were transduced with retroviruses expressing oncogenic ras (HrasV12) upon expression of p53. p53 was produced intracellularly by suppressing the transcription of a short-hairpin RNA directed against the natural p53 transcript by the administration of the antibiotic doxycycline. It was shown that expression of p53 resulted in the activation of senescence in tumour cells, and in the involution of the tumour by the innate immune system. Novellino et al, Cancer Immunol. Immunother. (2005) 54: 187-207, list tumour specific antigens.

### Objects of the invention

It is an object of the present invention to provide for pharmaceutical compositions suitable for use against malignant cells.

### General Description of the Invention

The invention achieves the above-mentioned objects by the subject-matter defined in the claims, and especially by providing the compounds for use as a medicament for use in the treatment of pre-malignant and/or of malignant cells, especially for inducing an immune response specifically directed against the malignant cells, the use of compounds for the production of pharmaceutical compositions suitable for use against cells expressing certain antigens, which for the present invention provide malignant cells, e.g. tumour cells and pre-malignant cells, as well as infected cells, e.g. cells containing an infecting virus or bacterium, especially chronically infected cells.

Generally, the present invention provides the use of compositions and pharmaceutical compositions for use as a medicament in the treatment of malignant cells by providing or inducing an adapted immune response, preferably including both cytotoxic T-cells and antibody producing cells, e.g. B-cells, which are specifically directed against malignant cells and premalignant cells. The compositions of the invention achieve the generation of a cellular and/or antibody-based immune response which is specifically directed against malignant cells by inducing immune cells, including professional antigen presenting cells for specificity against malignant cells by contacting immune cells with senescent premalignant or malignant cells.

Senescence of cells can generally be described by the following features of cells: A flattened morphology, optionally showing two or more nuclei, staining for SA-β-Gal (senescence associated β-galactosidase) positive, at least one immunohistochemical marker positive from the group of p19, p21, p53, p16, DCR2, DEK1, a reduced metabolic activity, and further optionally their resistance against the induction of apoptosis. According to the invention, compositions inducing the senescence of malignant cells can be provided to generate senescent malignant and premalignant cells in contact with the immune system, as it has been shown by the present inventors that senescent cells efficiently induce the generation of a specific immune response, including T-cells and antibody producing cells, e.g. B-cells, i.e. a cellular and humoral immune response, which is specifically directed against the senescent cell, e.g. against the antigen characterizing the malignant cell. Currently, it is assumed that by a bystander effect, the specific immune response raised is also directed against non-senescent cells expressing the same antigen, which would normally not be sufficient to induce an immune response without the interconnection of induction of an antigen specific immune response via senescent cells. In detail, the immune specificity of T-cells and B-cells which is elicited by contacting immune cells, preferably including professional antigen presenting cells (APC) with senescent malignant or premalignant cell, which is e.g. a virally or bacterially infected cell, is directed against antigen presented by MHC I and/or MHC II of the cell, e.g of the senescent cell. Preferably, pre-malignant cells are chronically infected cells, e.g. cells having a chronic viral or bacterial infection, e.g. by a hepatitis virus.

Accordingly in another embodiment, the compounds of the invention comprise senescent malignant cells for use in the production of a pharmaceutical composition for the medical use of generation of a specific immune response, which immune response is directed against an antigen of the malignant cell. Accordingly, the premalignant or malignant homologous senescent cell, e.g. prepared from a homologous or autologous premalignant or malignant cell obtained from the patient or animal by contacting the cell with a senescence inducing agent, can be used as a medicament, e.g. in tumour therapy, especially for inducing a specific immune response against the premalignant or malignant cell. The specific immune response induced by administration of autologous or homologous senescent premalignant or malignant cells comprises the cellular immune response, e.g. T-cells, and antibody production, e.g. antigen-specific B-cells. The antigen characterizing the malignant or pre-malignant cell can be a tumor-specific antigen, or a viral antigen or a bacterial antigen contained in the malignant or pre-malignant cell. In case that a pathogen (e.g. virus or bacterium) has been phagocytosed by an innate immune cell, this cell is referred to as a malignant cell, too, and will be a malignant cell in the terms of the invention that is subjected to senescence induction. For the purposes of the invention, the terms malignant and pre-malignant can be used interchangeably.

As the immune response, which is specifically raised and directed against malignant cells, comprises antigen-specific T-cells and/or B-cells producing antigen-specific antibody, the compounds of the invention can be used to generate an adapted, i.e. specific immune response comprising both cellular and humoral immune responses directed against cells which via MHC present the specific antigen, including cells in their active cell cycle, e.g. non-senescent cells, like actively proliferating tumour cells and infected cells.

In a first embodiment, the invention provides the use of an agent suitable for inducing senescence in non-senescent malignant cells for the production of a pharmaceutical composition for medical use against malignant cells, which are e.g. tumour cells or infected cells. The senescence inducing agent, e.g. a cytostatic agent or a chemotherapeutical agent or irradiation, especially radioactive irradiation, overexpression of p53 or p14Arf, or Nutlin, especially the MDM2-inhibitor Nutlin-3 ((+/−)-4-[4,5-Bis(4-chlorophenyl)-2-(2-isopropoxy-4-methoxyphenyl)-4,5-dihydroimidazol-1-carbonyl]-piperazin-2-on), and/or a PTEN-inhibitor, e.g. VO-OHpic is used to contact non-senescent malignant and/or premalignant cells in vivo or in vitro for the generation of senescent malignant cells, which are contacted with immune cells in vitro or in vivo, i.e. in the body of the patient. By the contact with the senescent malignant cells, the immune cells, preferably including APC, are induced to produce a specific immune response directed against the malignant cells. In an in vivo application, the pharmaceutical composition has a dosage effective to induce senescence in at least a fraction of malignant or premalignant cells within a patient, preferably at a non-cytotoxic dosage, e.g. by administration of a the senescence-inducing agent at an effective dosage less than the dosage required for inducing apoptosis, e.g. less than the dosage required for IC 50.

Accordingly, the invention in this embodiment provides senescence inducing compounds for use as a medicament for the treatment of malignant cells by induction of a cellular and humoral immune response that is specific for the malignant cells, e.g. specifically directed against an antigen characterizing the malignant cells. Accordingly, the senescence inducing compounds in this embodiment are adapted or prepared for inducing the senescence of malignant cells, which in turn induces a specific immune response against the malignant cells. Generally, such compounds include cytostatic compounds at a dosage sufficient to induce senescence of malignant cells in vivo, which dosage is sufficiently low to essentially not impair the immune system. Such a dosage could be in a range of 10 to 50%, preferably at 15 to 30% of the dosage of chemotherapy and could e.g. be used for the treatment of patients having a general health status that would sustain full chemotherapy.

Preferably, the provision of senescent malignant cells within a patient generated from non-senescent malignant cells by administration of a pharmaceutical composition containing a senescence-inducing agent, is in combination with the administration of an immuno-stimulant in order to enhance the activity of the immune system, preferably enhancing the activity of APC and T-cells. Accordingly, the invention also provides for the use of an immuno-stimulating agent for use as a medicament and/or for the production of a pharmaceutical composition for use against malignant cells, especially in combination with the use of a senescence-inducing compound as a medicament and/or for the production of a pharmaceutical composition, as well as providing for a pharmaceutical composition comprising a senescence-inducing compound in combination with an immuno-stimulating agent or compound for use as a medicament for use in the treatment of malignant cells. In the first embodiment, the pharmaceutical composition is used as a medicament, e.g. for use in the treatment of malignant cells, to generate senescent malignant cells within a patient in order to induce the generation of a specific immune response, preferably comprising both specific T-cells and B-cells generating specific antibody, which is directed against at least one antigen of the malignant cell. For enhancing the generation of the specific cellular and/or specific humoral immune response directed against the antigen of the malignant cell, an immuno-stimulating agent can be administered, preferably in combination with the pharmaceutical composition containing the senescence-inducing compound. Accordingly, the use of a senescence-inducing compound for the production of a pharmaceutical composition for use against malignant cells is also provided in combination with the use of a immuno-stimulating agent for the production of the pharmaceutical composition for use against malignant cells.

In a second embodiment, the specific immune response, especially T-cells which are specifically directed against the malignant cells (or antibodies produced by B-cells), is generated from autologous immune cells of the patient in vitro by contacting autologous immune cells of the patient with senescent malignant cells, especially homologous or autologous malignant cells of the patient or senescent non-malignant cells overexpressing an antigen. In this embodiment, the invention provides for the use of homologous, preferably autologous malignant or non-malignant cells of a patient as a medicament and/or for the production of a pharmaceutical composition for use in the medical treatment against malignant cells, especially against tumour cells and/or infected cells, the pharmaceutical composition comprising senescent homologous malignant cells, preferably senescent autologous malignant or premalignant cells of the patient in a formulation for implantation into the patient, e.g. by formulating the senescent autologous malignant cells for injection into the patient. In this embodiment, senescent malignant cells for use as a medicament for use in the treatment of malignant cells, which are e.g. tumour cells and/or chronically infected cells, and/or in the production of the pharmaceutical composition can be generated from isolated autologous malignant cells or non-malignant cells by contacting with a senescence-inducing agent, e.g. by contacting autologous malignant cells with a cytostatic agent or radioactive irradiation at a dosage below the dosage required for inducing apoptosis, e.g. below the dosage corresponding to the IC50 or by using other senescence inducing compounds as described below. Malignant cells can be obtained from the patient's tumour (to be then subjected to a senescence inducing agent) or generated by in vitro genetic manipulation, e.g. by transient or permanent transformation, of homologous cells, preferably autologous cells of the patient with a nucleic acid construct encoding an antigen against which an immune response is desired, expressing the antigen in the genetically manipulated cells, and inducing senescence. The genetic manipulation of cells of the patient is preferred for a heterologous antigen, e.g. for use in the treatment of chronic infections (e.g. the heterologous antigen selected from viral or bacterial protein) or for homologous antigen characteristic of a disease like a tumour antigen (e.g. MUC1), to produce senescent malignant cells characterized by the antigen, as these cells will induce an effective immune response after re-introduction into the patient. Alternative methods for senescence induction are, as described, overexpression of certain oncogenes (e.g. oncogenic Ras) or overexpression of tumour suppressor genes, e.g. p14Arf or p53, or contacting with MDM2-inhibitor, e.g. Nutlin, or contacting with a PTEN-inhibitor, e.g. VO-OHpic.

In this embodiment, the pharmaceutical composition of the invention comprises senescent autologous malignant cells for use in the medical treatment of malignant cells, e.g. senescent tumour cells or infected cells, especially for use as a medicament, in a formulation for injecting the senescent malignant cells into the patient. This embodiment makes use of the observation that senescent malignant or premalignant cells potently induce a specific cellular and/or humoral immune response, specifically directed against the antigen presented by the senescent malignant cell. Accordingly, in this and other embodiments it is preferred that the pharmaceutical composition comprises an immuno-stimulating agent, e.g. an interferon and/or cytokine for general activation of the immune system. Optionally, senescent pre-malignant or malignant cells containing the antigen against which the induction of a specific immune response is desired can be generated by genetic manipulation of autologous/homologous or also heterologous cells (i.e. from other individuals), e.g. in vitro using cells obtained from a patient, for introduction of an expression cassette encoding an antigen against which a specific immune response is desired, followed by inducing senescence of the cells. As an example, autologous cells can be nucleated cells, e.g. fibroblasts, which after genetic manipulation in vitro for expression of the antigen and after induction of senescence are used as a medicament for use in the treatment against cells within the patient for treatment against malignant cells expressing the same antigen. In a further embodiment, the senescent malignant cells obtained by in vitro manipulation for expression of the antigen (e.g. by introduction of an expression cassette encoding the antigen into the autologous cells in vitro) are contacted in vitro with un-primed immune cells obtained from the patient for priming the immune cells for specificity against the antigen. Priming of the immune cells against the antigen can either occur in vivo in the patient (after administration of the cells into the patient, or, alternatively, the immune cells can be primed by in vitro contacting with senescent cells expressing the antigen, e.g. malignant cells, and are then used as a medicament, especially for use in the treatment of cells expressing the antigen, e.g. in the treatment of malignant cells which are characterized by expressing the antigen. Alternatively, a patient's tumour cells expressing a certain tumour antigen can be isolated, subjected to senescence induction outside the patient and then be re-transplanted into the patient to induce an antigen specific immune response against the residual tumour cells.

In a third embodiment, the invention provides for the use of homologous, especially of autologous immune cells for the production of a pharmaceutical composition for the medical treatment of malignant cells, especially tumour cells and infected cells in a patient, wherein the immune cells are specific for the malignant cell. For generating immune cells having specificity for malignant cells, homologous and preferably autologous immune cells of a patient are in vitro contacted with malignant cells of the patient in which malignant cells ex vivo, e.g. in vitro, senescence has been induced. Generally, the immune cells are preferably homologous or autologous to the patient. Alternatively, normal cells from a patient (e.g. skin fibroblasts) can be taken in culture, an antigen of choice (e.g. a viral antigen, a bacterial antigen or a tumour antigen) will be delivered into these cells and subsequently senescence will be induced in these cells to generate senescent malignant cells. Antigen presenting cells and adaptive immune cells can then be contacted with these cells to induce antigen specific immune cells. In this embodiment, a pharmaceutical composition for the treatment of malignant cells in a patient can be generated by contacting autologous immune cells, which are e.g. isolated from the patient, with senescent malignant cells, as it has been found that the presence of senescent malignant cells in contact with immune cells leads to the generation of a specific immune response of the immune cells directed against the senescent malignant cells. As described in the other embodiments of the invention, senescent malignant cells can be generated by inducing senescence in malignant cells, e.g. in homologous or autologous immune cells derived from the patient by contacting with a senescence—inducing agent, e.g. a cytostatic agent or irradiation at a dosage below the dosage inducing apoptosis or by gene transfer or by other senescence inducing compounds as described below. Preferably, in this embodiment the pharmaceutical composition comprising cells specifically directed against malignant cells by contacting the immune cells with senescent malignant cells is a formulation suitable for implantation of the immune cells into the patient.

As described in relation to the second embodiment, a senescent malignant cell can be generated from an autologous cell of the patient by direct in vitro contacting with a senescence inducing agent or by genetic manipulation with a nucleic acid sequence encoding an antigen, e.g. a homologous or a heterologous antigen specific for a tumour, a virus or bacterium, and subsequent senescence induction. The in vitro generation of immune cells specifically directed against senescent and non-senescent cells having the antigen is by contacting immune cells with the genetically manipulated senescent cells expressing the antigen, preferably followed by isolating antigen-specific primed immune cells, most preferably followed by isolating antigen-specific primed immune cells and expanding these, optionally with subsequent integration of these cells into a formulation suitable for introduction into the patient.

In a fourth embodiment, the invention relates to a process for producing immune cells specific for an antigen contained in a malignant cell, e.g. for producing B-cells and/or T-cells specifically directed against a malignant cell. In this embodiment, the invention also relates to a process for producing antibody by cultivating B-cells which have been generated to produce antibody specifically directed against an antigen presented by the malignant cell. The process comprises the step of introducing an antigen, against which immune specificity is to be generated in a cell, e.g. by transforming a cell with a nucleic acid construct comprising a sequence encoding the antigen, preferably in an expression cassette. The cells containing the antigen, which are for the purposes of this invention also related to as malignant cells, are subject to a treatment to induce their senescent state, i.e. by contacting with a senescence inducing agent to generate senescent malignant cells, which contain, and preferably express the antigen. In this embodiment, the malignant cells are cells that are genetically manipulated to express the antigen against which an immune response is desired, including one or both of cellular immune response and antibody production. These senescent, antigen-containing cells are then contacted with immune cells, preferably B-cells and/or T-cells, most preferably in the presence of APC. Using isolation of cells and identification of cells having the specificity against the antigen, especially B-cells producing an antibody specific for the antigen by standard immuno assays. For antigen-specific T-cells, it is preferred to use an Elispot assay for identification, for antibody producing B-cells, an ELISA can be used. Preferably, the cells subjected to the introduction of an expression cassette encoding the antigen are autologous cells of the experimental animal, and more preferably, these genetically manipulated cells are subjected to induction of senescence in vitro and then transferred back into the animal.

For the production of antibody, especially for the production of monoclonal antibody, it is preferred to fuse the B-cell producing the antibody against the antigen using hybridoma technique, and cultivating the resultant antibody producing hybridoma.

In the fourth embodiment, the process for producing antigen-specific immune cells, especially for producing antibody, can be using an experimental animal, e.g. a mouse, rats, goat, sheep, by transforming cells, especially spleen cells with a nucleic acid construct expressing the antigen, e.g. using a viral vector containing an expression cassette encoding the antigen, and inducing senescence in at least a fraction of the transformed cells, e.g. by application of a senescence-inducing agent, followed by isolation of spleen cells. From the isolated spleen cells, an immune cell can be isolated having the specificity against the antigen used, especially selecting a B-cell producing an antibody specific for the antigen as described above, preferably followed by generating a hybridoma using fusion with a tumour cell, followed by cultivation of the hybridoma for producing the antibody.

In greater detail, the invention provides evidence that induction of a specific immune response is caused by contacting of the immune cells with malignant cells, e.g. tumour cells, virally or bacterially infected cells or cells expressing an antigen. The strong immune response induced by presence of the senescent cells presenting antigen was shown on the example of normal epithelial cells, represented by liver cells, which express oncogenic ras, in the state of senescence. For the purposes of this invention, the term “malignant cell” relates to an animal or human cell, in case of pharmaceutical compositions of the invention preferably to homologous/autologous cells of the patient or heterologous cells, which express an antigen, either due to malignancy of the cell, as in the case of tumour cells, and in the case of purposes of antibody production, the term “malignant cell” also relates to cells which are transformed with a coding sequence encoding an antigen for expressing the antigen, preferably an antigen heterologous to the cell.

In the following detailed section, experimental proof is provided for the observation that innate immune cells as well as adapted immune cells are infiltrating in the tissue upon induction of senescence in malignant cells, e.g. infiltrate the liver upon induction of senescence in malignant cells. Various control experiments demonstrate that an antigen specific immune response, the antigen characterising the malignancy of the malignant cells, is raised by presence of senescent malignant cells, including senescent pre-malignant cells. The antigen-specific immune response directed against the senescent malignant and pre-malignant cells is sufficient for clearance of malignant and pre-malignant cells, both in their senescent and in their non-senescent state. A defect in the adaptive immunity pathway, e.g. the genetic SCID defect (defects in function of T-lymphocytes, B-lymphocytes, and of NKT-cells), completely abrogates the generation of antigen-specific immune cells, whereas IFN-gamma-Elispot analysis demonstrates presence of antigen-specific T-cells which are directed against the factor which determines malignancy of the malignant senescent cells, which is in the present case represented by the exemplary oncogenic Nras, e.g. in a non-defective immune system. The finding that an Arf −/− the genotype, which bypasses the induction of senescence in malignant cells, prevents the induction of a specific T-cell response demonstrates that it is the presence of senescent malignant cells which induces the antigen-specific immune response.

Preferably, the malignant cell is characterized by presenting a tumor-specific antigen, which accordingly is not expressed by non-malignant and not by non-premalignant cells. Tumor-specific antigen include those which are e.g. mentioned in Novellino et al, Cancer Immunol. Immunother. (2005) 54: 187-207, which is included herein by reference in respect of tumour specific antigens. Preferably, a tumour specific antigen is one of the group comprising or consisting of tumour antigens resulting from mutations, shared tumour antigens, differentiation antigens, antigens overexpressed in tumours, especially Nras, Hras, Kras which are indicative of a neoplastic state, tumour-specific antigens of the MAGE (including MAGE-B5, MAGE-B6, MAGE, MAGE-C2, MAGE-C3, MAGE-D), HAGE, SAGE, SSX-2, BAGE, TRAG-3, and GAGE families, including NY-ESO-1, LAGE, CAMEL, as well as MUC1, most preferably tumour-specific mutant Ras, e.g. Nras, Nras G12V, or Kras, KrasG12D and other common mutations.

Exemplary tumour antigens resulting from mutations are for lung carcinoma FIASNGVKLV, for melanoma YSVYFNLPADTIYTN, for chronic myeloid leukemia SSKALQRPV or GFKQSSKAL or ATGFKQSSKALQRPVAS or ATGFKQSSKALQRPVAS, for melanoma EDLTVKIGDFGLATEKSRWSGSHQFEQLS, for colorectal, gastric, and endometrial carcinoma FLIIWQNTM, for head and neck squamous cell carcinoma FPSDSWCYF, for melanoma SYLDSGIHF, for melanoma FSWAMDLDPKGA, for melanoma ACDPHSGHFV, for melanoma AVCPWTWLR, for colorectal carcinoma TLYQDDTLTLQAAG or TLYQDDTLTLQAAG, for myeloid leukemia TMKQICKKEIRRLHQY, for melanoma KILDAVVAQK, for lung squamous CC especially ETVSEQSNV, for acute lymphoblastic leukemia RIAECILGM or IGRIAECILGMNPSR or IGRIAECILGMNPSR, for acute myelogenous leukemia YVDFREYEYY, for melanoma MIFEKHGFRRTTPP, for melanoma TLDWLLQTPK, for melanoma WRRAPAPGA or PVTWRRAPA, for renal cell carcinoma, for melanoma and renal cell carcinoma SLFEGIDIYT, for bladder tumour AEPINIQTW, for melanoma FLEGNEVGKTY, for non-small cell lung carcinoma FLDEFMEGV, for melanoma EEKLIVVLF, for melanoma SELFRSGLDSY or FRSGLDSYV, for melanoma EAFIQPITR, for melanoma RVIKNSIRLTL, for melanoma KINKNPKYK, lung squamous cell carcinoma QQITKTEV, colorectal carcinoma SLYKFSPFPL, for melanoma KELEGILLL, for head and neck squamous cell carcinoma VVPCEPPEV, for promyelocytic leukemia NSNHVASGAGEAAIETQSSSSEEIV, for melanoma LLLDDLLVSI, for melanoma PYYFAAELPPRNLPEP, for pancreatic adenocarcinoma VVVGAVGVG, for melanoma ILDTAGREEY, for melanoma RPHVPESAF, for melanoma KIFSEVTLK, for melanoma SHETVIIEL, for sarcoma QRPYGYDQIM, for colorectal carcinoma RLSSCVPVA, for melanoma GELIGILNAAKVPAD.

Exemplary shared tumour antigens are:

AARAVFLAL, YRPRPRRY, YYWPRPRRY, VLPDVFIRC(V), MLAVISCAV, RQKRILVNL, NYNNFYRFL, EYSKECLKEF, EYLSLSDKI, MLMAQEALAFL, SLLMWITQC, LAAQERRVPR, ELVRRILSR, APRGVRMAV, SLLMWITQCFLPVF, QGAMLAAQERRVPRAAEVPR, AADHRQLQLSISSCLQQL, CLSRRPWKRSWSAGSCPGMPHL, CLSRRPWKRSWSAGSCPGMPHL, ILSRDAAPLPRPG, AGATGGRGPRGAGA, EADPTGHSY, KVLEYVIKV, SLFRAVITK, EVYDGREHSA, RVRFFFPSL, EADPTGHSY, REPVTKAEML, DPARYEFLW, ITKKVADLVGF, SAFPTTINF, SAYGEPRKL, SAYGEPRKL, TSCILESLFRAVITK, PRALAETSYVKVLEY, FLLLKYRAREPVTKAE, EYVIKVSARVRF, YLQLVFGIEV, EYLQLVFGI, REPVTKAEML, EGDCAPEEK, LLKYRAREPVTKAE, EVDPIGHLY, FLWGPRALV, KVAELVHFL, TFPDLESEF, VAELVHFLL, MEVDPIGHLY, EVDPIGHLY, REPVTKAEML, AELVHFLLL, MEVDPIGHLY, WQYFFPVIF, EGDCAPEEK, KKLLTQHFVQENYLEY, KKLLTQHFVQENYLEY, ACYEFLWGPRALVETS, VIFSKASSSLQL, VIFSKASSSLQL, GDNQIMPKAGLLIIV, TSYVKVLHHMVKISG, RKVAELVHFLLLKYRA, FLLLKYRAREPVTKAE, EVDPASNTY, GVYDGREHTV, NYKRCFPVI, SESLKMIF, MVKISGGPR, EVDPIGHVY, REPVTKAEML, EGDCAPEEK, ISGGPRISY, LLKYRAREPVTKAE, ALSVMGVYV, GLYDGMEHL, DPARYEFLW, FLWGPRALV, VRIGHLYIL, EGDCAPEEK, REPFTKAEMLGSVIR, AELVHFLLLKYRAR, LLFGLALIEV, ALKDVEERV, SESIKKKVL, PDTRPAPGSTAPPAHGVTSA, QGQHFLQKV, SLLMWITQC, MLMAQEALAFL, ASGPGGGAPR, LAAQERRVPR, TVSGNILTIR, APRGPHGGAASGL, MPFATPMEA, KEFTVSGNILTI, MPFATPMEA, LAMPFATPM, ARGPESRLL, SLLMWITQCFLPVF, LLEFYLAMPFATPMEAELARRSLAQ, LLEFYLAMPFATPMEAELARRSLAQ, EFYLAMPFATPM, RLLEFYLAMPFA, QGAMLAAQERRVPRAAEVPR, PGVLLKEFTVSGNILTIRLT, VLLKEFTVSG, AADHRQLQLSISSCLQQL, LLEFYLAMPFATPMEAELARRSLAQ, LKEFTVSGNILTIRL, PGVLLKEFTVSGNILTIRLTAADHR, LLEFYLAMPFATPMEAELARRSLAQ, AGATGGRGPRGAGA, LYATVIHDI, ILDSSEEDK, KASEKIFYV, EKIQKAFDDIAKYFSK, WEKMKASEKIFYVYMKRK, KIFYVYMKRKYEAMT, KIFYVYMKRKYEAM, INKTSGPKRGKHAWTHRLRE, YFSKKEWEKMKSSEKIVYVY, MKLNYEVMTKLGFKVTLPPF, KHAWTHRLRERKQLVVYEEI, LGFKVTLPPFMRSKRAADFH, KSSEKIVYVYMKLNYEVMTK, KHAWTHRLRERKQLVVYEEI, SLGWLFLLL, LSRLSNRLL, LSRLSNRLL, CEFHACWPAFTVLGE, CEFHACWPAFTVLGE, CEFHACWPAFTVLGE, EVISCKLIKR or CATWKVICKSCISQTPG.

Exemplary tumour differentiation antigens are:

YLSGANLNL, IMIGVLVGV, GVLVGVALI, HLFGYSWYK, QYSWFVNGTF, TYACFVSNL, AYVCGIQNSVSANRS, DTGFYTLHVIKSDLVNEEATGQFRV, YSWRINGIPQQHTQV, TYYRPGVNLSLSC, EIIYPNASLLIQN, YACFVSNLATGRNNS, LWWVNNQSLPVSP, LWWVNNQSLPVSP, LWWVNNQSLPVSP, EIIYPNASLLIQN, NSIVKSITVSASG, KTWGQYWQV, (A)MLGTHTMEV, ITDQVPFSV, YLEPGPVTA, LLDGTATLRL, VLYRYGSFSV, SLADTNSLAV, RLMKQDFSV, RLPRIFCSC, LIYRRRLMK, ALLAVGATK, IALNFPGSQK, ALNFPGSQK, ALNFPGSQK, VYFFLPDHL, RTKQLYPEW, HTMEVTVYHR, SSPGCQPPA, VPLDCVLYRY, LPHSSSHWL, SNDGPTLI, GRAMLGTHTMEVTVY, WNRQLYPEWTEAQRLD, TTEWVETTARELPIPEPE, TGRAMLGTHTMEVTVYH, GRAMLGTHTMEVTVY, SVSESDTIRSISIAS, LLANGRMPTVLQCVN, RMPTVLQCVNVSVVS, PLLENVISK, (E)AAGIGILTV, ILTVILGVL, EAAGIGILTV, AEEAAGIGIL(T), RNGYRALMDKS, EEAAGIGILTVI, AAGIGILTVILGVL, APPAYEKLpSAEQ, EEAAGIGILTVI, RNGYRALMDKSLHVGTQCALTRR, MPREDAHFIYGYPKKGHGHS, KNCEPVVPNAPPAYEKLSAE, SLSKILDTV, LYSACFWWL, FLTPKKLQCV, VISNDVCAQV, VLHWDPETV, MSLQRQFLR, ISPNSVFSQWRVVCDSLEDYD, SLPYWNFATG, SVYDFFVWL, TLDSQVMSL, LLGPGRPYR, LLGPGRPYR, ANDPIFVVL, QCTEVRADTRPWSGP, ALPYWNFATG, KCDICTDEY, SSDYVIPIGTY, MLLAVLYCL, CLLWSFQTSA, YMDGTMSQV, AFLPWHRLF, QCSGNFMGF, TPRLPSSADVEF, LPSSADVEF, LHHAFVDSIF, SEIWRDIDF, QNILLSNAPLGPQFP, SYLQDSDPDSFQD or FLLHHAFVDSIFEQWLQRHRP.

Exemplary antigens overexpressed in tumour are:

SVASTITGV, RSDSGQQARY, LLYKLADLI, YLNDHLEPWI, CQWGRLWQL, VLLQAGSLHA, KVHPVIWSL, LMLQNALTTM, LLGATCMFV, NPPSMVAAGSVVAAV, ALGGHPLLGV, TMNGSKSPV, RYQLDPKFI, DVTFNIICKKCG, FMVEDETVL, FINDEIFVEL, KYDCFLHPF, KYVGIEREM, NTYASPRFK, HLSTAFARV, KIFGSLAFL, IISAVVGIL, ALCRWGLLL, ILHNGAYSL, RLLQETELV, VVLGVVFGI, YMIMVKCWMI, HLYQGCQVV, YLVPQQGFFC, PLQPEQLQV, TLEEITGYL, ALIHHNTHL, PLTSIISAV, VLRENTSPK, TYLPTNASL, ALLEIASCL, WLPFGFILI, SPRWWPTCL, GVALQTMKQ, FMNKFIYEI, QLAVSVILRV, LPAVVGLSPGEQEY, VGQDVSVLFRVTGALQ, VLFYLGQY, TLNDECWPA, GLPPDVQRV, SLFPNSPKWTSK, STAPPVHNV, LLLLTVLTV, PGSTAPPAHGVT, LLGRNSFEV, RMPEAAPPV, SQKTYQGSY, PGTRVRAMAIYKQ, HLIRVEGNLRVE, TLPGYPPHV, CTACRWKKACQR, VLDGLDVLL, SLYSFPEPEA, ALYVDSLFFL, SLLQHLIGL, LYVDSLFFL, NYARTEDFF, LKLSGVVRL, PLPPARNGGL, SPSSNRIRNT, LAALPHSCL, GLASFKSFLK, RAGLQVRKNK, ALWPWLLMA(T), NSQPVWLCL, LPRWPPPQL, KMDAEHPEL, AWISKPPGV, SAWISKPPGV, MIAVFLPIV, HQQYFYKIPILVINK, ELTLGEFLKL, ILAKFLHWL, RLVDDFLLV, RPGLLGASVLGLDDI, LTDLQPYMRQFVAHL, SRFGGAVVR, TSEKRPFMCAY, CMTWNQMNL, LSHLQMHSRKH or KRYFKLSHLQMHSRKH.

In the case of malignant cells being bacterially infected or virally infected cells, the malignant cells are preferably characterized by presenting a bacterial or viral antigen, respectively. Exemplary bacterial antigen are e.g. antigens originating from Staphylococcus, Streptococcus, Enterococcus, Corynebacterium spec., Bacillus spec., Listeria spec., Clostridium spec., Mycobacterium spec., Actinomyces spec., Nocardia spec., Enterobacteriaceae, Escherichia spec., Proteus spec., Klebsiella spec., Serratia spec., Enterobacter spec., Salmonella, Shigella, Salmonella spec., Shigella spec., Pseudomonas, Vibrio spec., Campylobacter spec., Bacteriodes fragilis, Neisseria spec., Haemophilus spec., Bordetella spec., Brucella spec., Legionella, Spirochaetales spec., Mykoplasma, Rickettsia, Chlamydia spec.

Exemplary viral antigens are e.g. antigens originating from Picornavirus spec., e.g. Poliovirus, Coxsackievirus, Echo- and Enterovirus, Hepatitis A Virus, Rhinovirus, Flavivirus spec., e.g. Yellow fever virus, Dengue virus, FSME virus, Hepatitis C virus, Togavirus spec., e.g. Togavirus, Rubella virus, Coronavirus spec., Calicivirus spec., e.g. Norwalk virus, Hepatitis E virus, Rhabdovirus spec., e.g. Rabies virus, Paramyxovirus spec., e.g. Parainfluenza virus, Mumps virus, Measeles virus, Respiratory Syncytialvirus, Filovirus spec., e.g. Marburg virus, Ebola virus, Bornavirus spec., Orthomyxovirus spec., e.g. Orthomyxovirus, Influenza virus, Bunyavirus spec., e.g. Hanta virus, Arenavirus spec., e.g. LCMV virus, Hemorrhagic fever virus, Reovirus spec., e.g. Rotavirus, Retrovirus spec., e.g. HIV virus, HTLV virus, Hepadnavirus spec., e.g. Hepatitis B virus, Hepatitis D virus, Papovavirus spec., e.g. Polyomavirus, BK- and JC virus, Papillomavirus, Adenovirus spec., e.g. Herpesvirus spec., e.g. Herpes simplex virus, Varicella zoster virus, Cytomegalovirus, Herpes virus 6 and 7, Epstein-Barr-Virus, Herpesvirus 8, Poxvirus spec., e.g. Variolavirus, Parvovirus spec., e.g. Parvovirus B19, Adenoassociated virus, a virus of the papilloma virus genus, and viral ras.

### DETAILED DESCRIPTION OF THE INVENTION

In the following description, the invention is described by way of an example with reference to the figures.

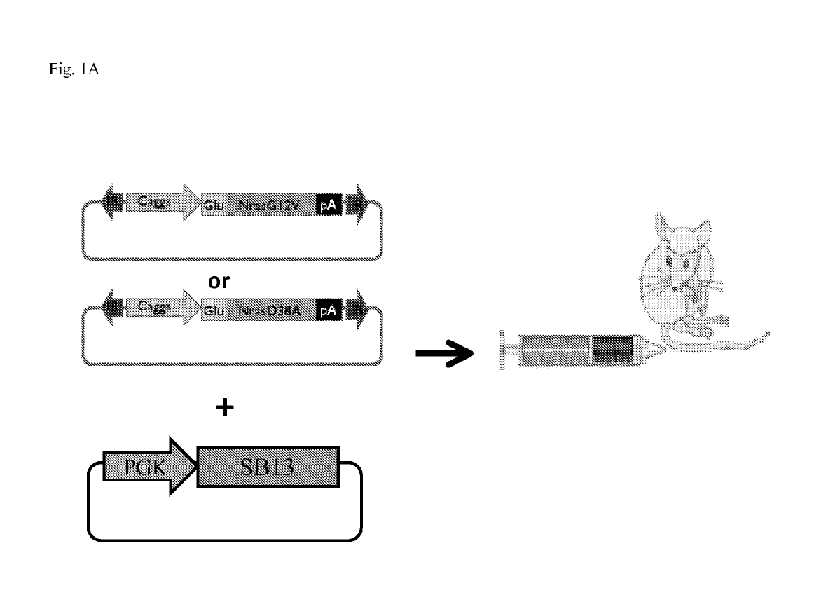

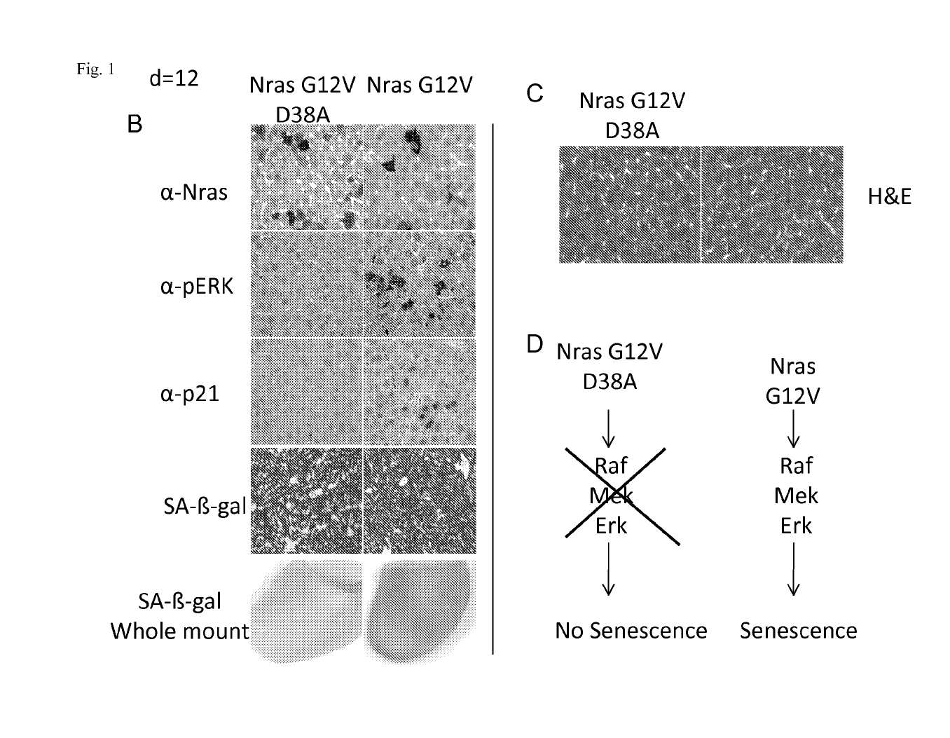

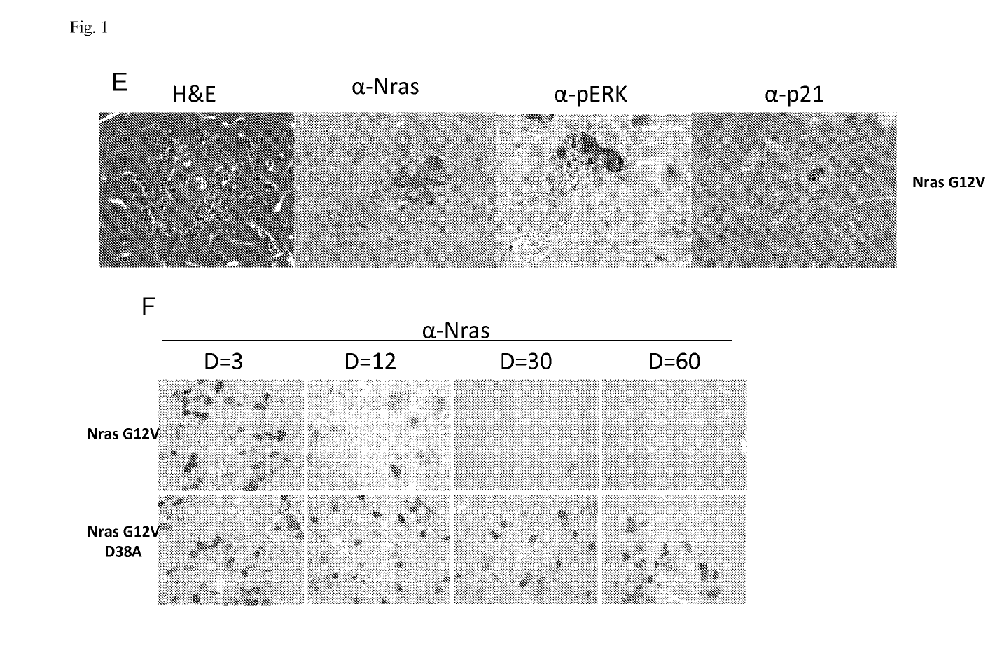

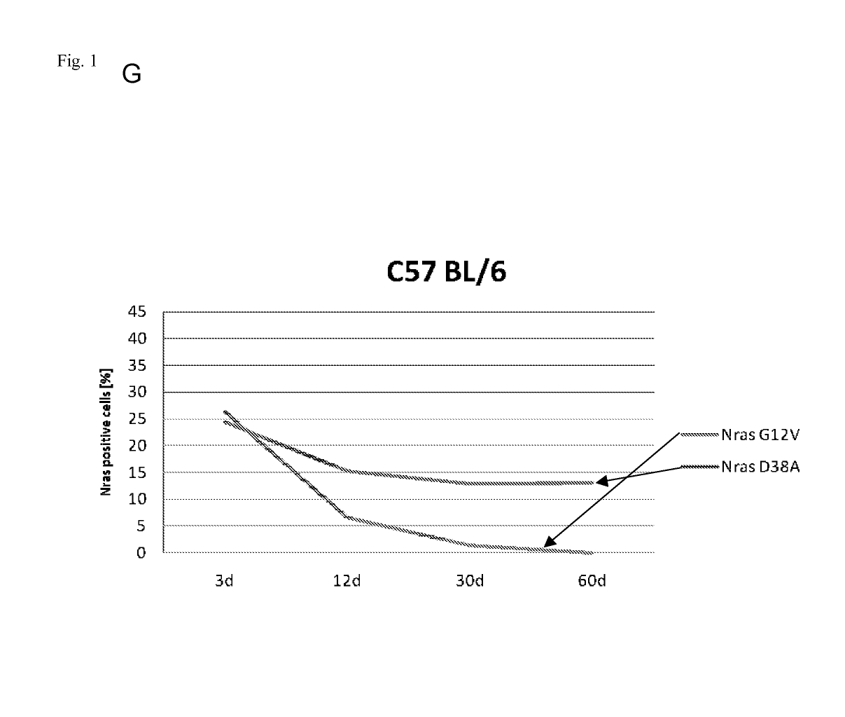

FIG. 1A schematically shows the generation of premalignant senescent cells by introducing an oncogene encoding expression cassette to cells (liver) of an experimental animal,

FIG. 1B shows micrographs and a photograph of livers cells of the experimental animals with specific stains,

FIG. 1C shows micrographs of H- and E-staining of liver cells of experimental animals,

FIG. 1D schematically describes the functional difference between the oncogene NrasG12V and it's kinase dead mutant Nras G12V-D38A.

FIG. 1E shows micrographs of livers from experimental animals, indicating that senescent premalignant cells are attacked by infiltrating immune cells.

FIG. 1F shows micrographs of the livers of experimental animals at day 3, 12, 30 and 60, respectively, following the expression of oncogene in cells.

FIG. 1G shows a graph of the quantification of the exemplary premalignant senescent cells in a 60 day time course. Non senescent cells expressing the kinase dead mutant D38A serve as a control.

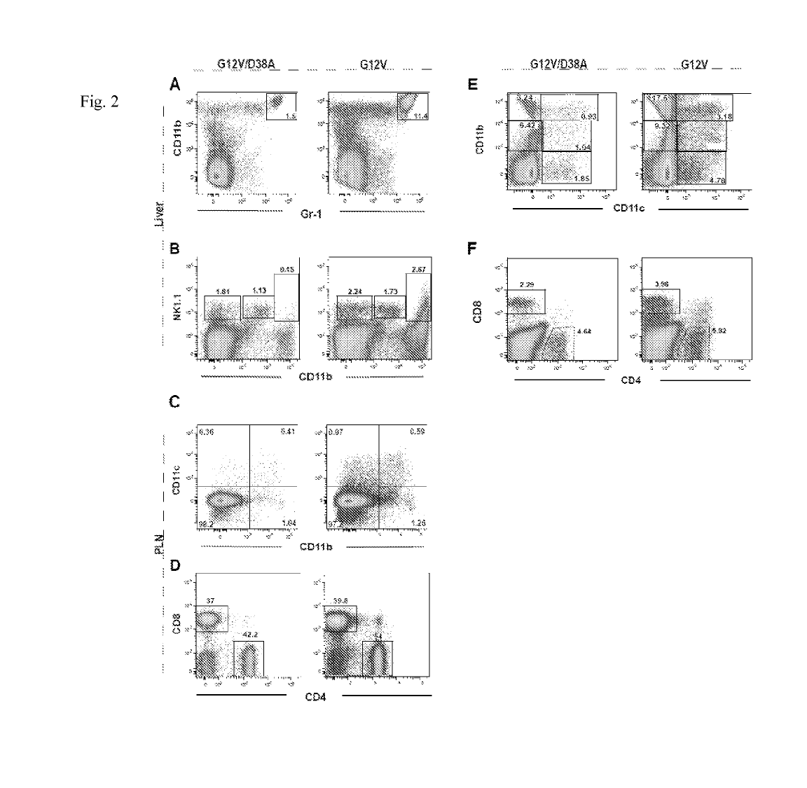

FIG. 2 shows measurement results of FACS analyses of single cell suspensions obtained from either the liver (liver) or the portal lymph node of the animals harbouring premalignant senescent cells in their livers As described, liver cells of experimental animals express G12V and the mutant G12V D38A, respectively. FACS analyses were performed using different antibodies directed against surface markers of immune cells.

namely in FIG. 2A antibody staining, CD11b and Gr-1 (Neutrophil Granulocytes) in FIG. 2B for NK1.1 and antibody staining for CD11b, for natural killer cells

FIG. 2C antibody staining for CD11 and CD11b for dendritic cells (here from portal lymph nodes (PLN)), and in FIG. 2D antibody staining for CD8 and CD4 positive lymphocytes found in PLN,

in FIG. 2E antibody staining for CD11b and CD11c (dendritic cells) found in the liver

in FIG. 2F with antibody staining for CD8 and CD4 positive immune cells from the liver

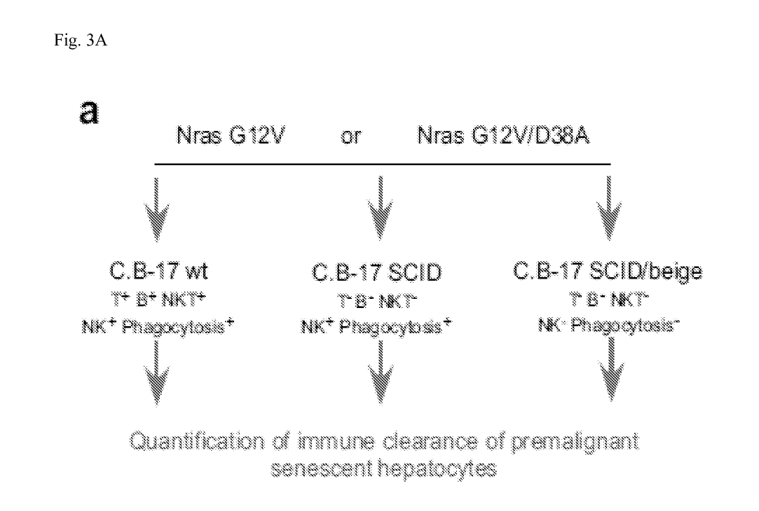

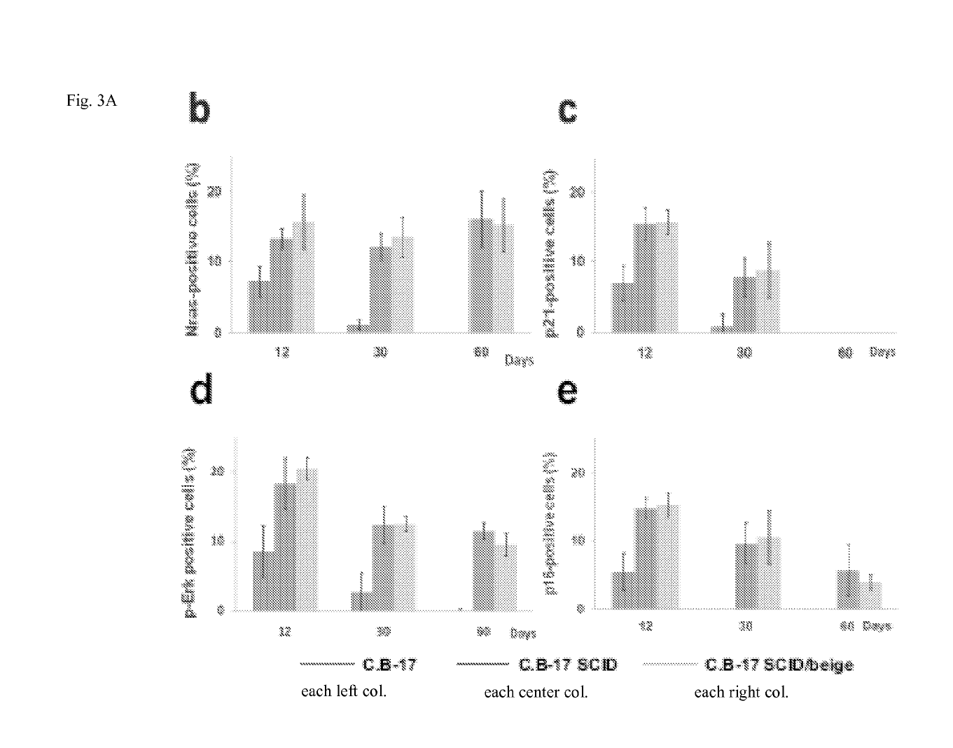

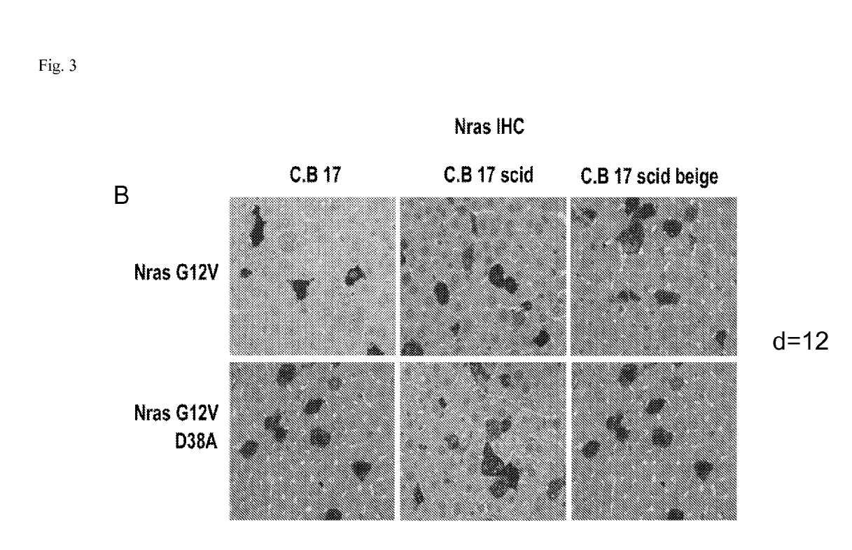

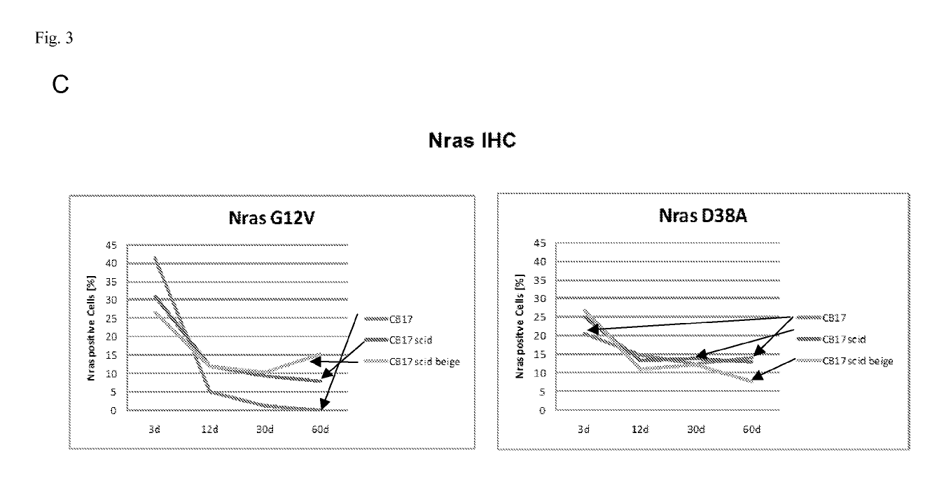

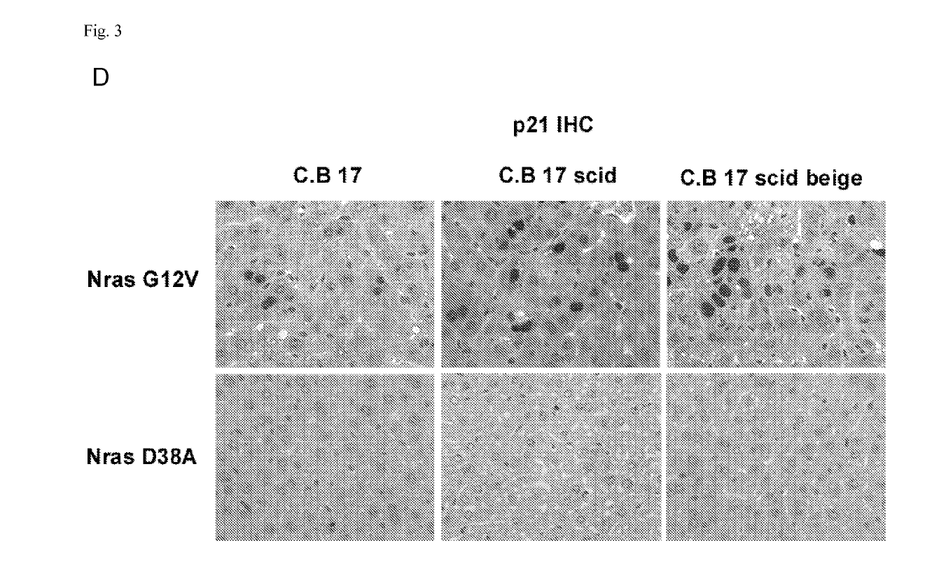

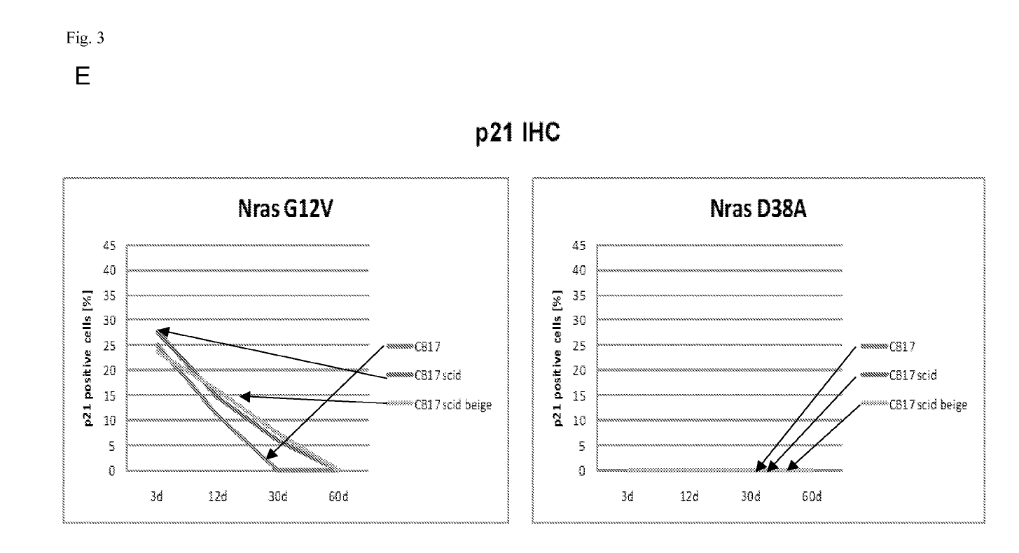

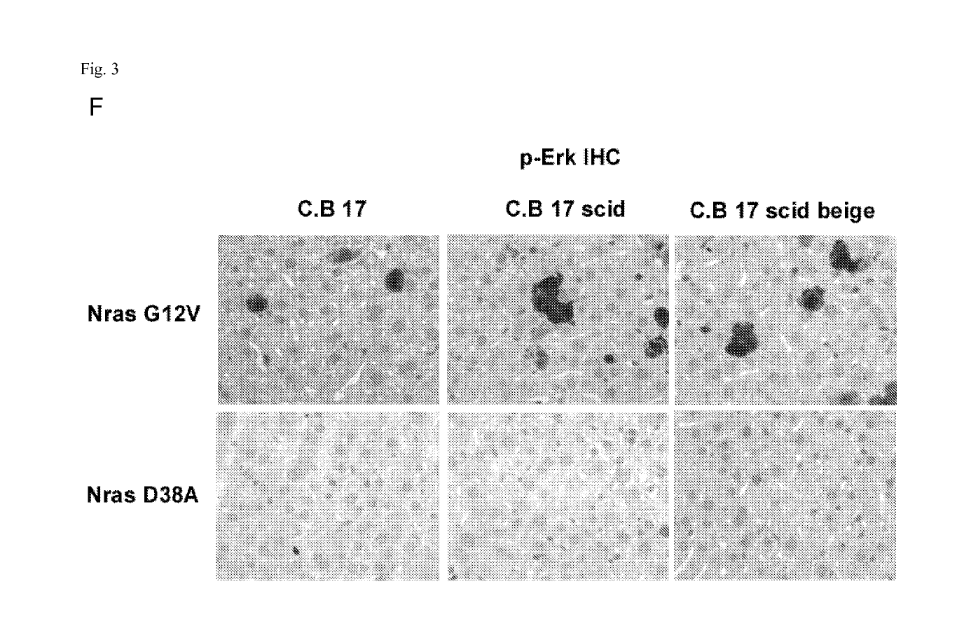

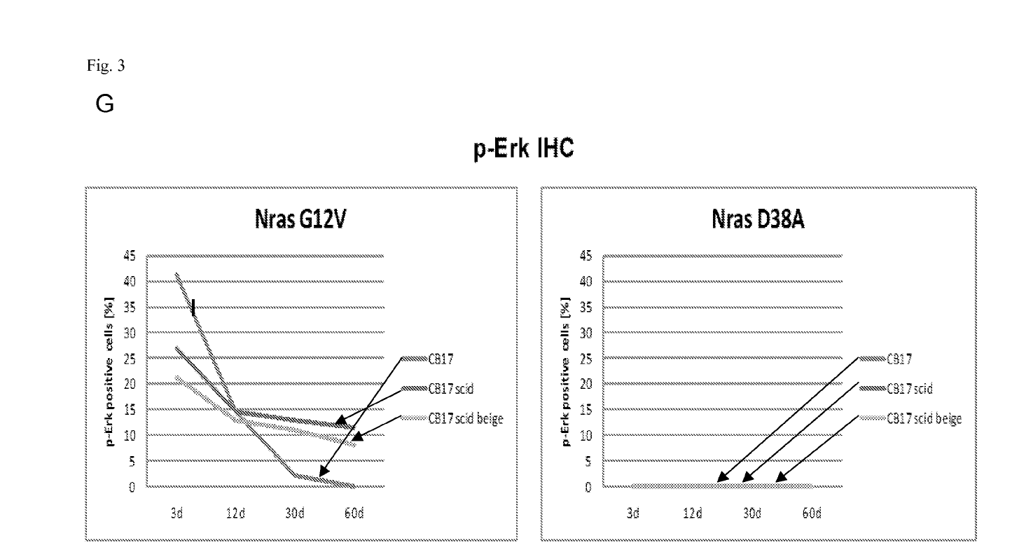

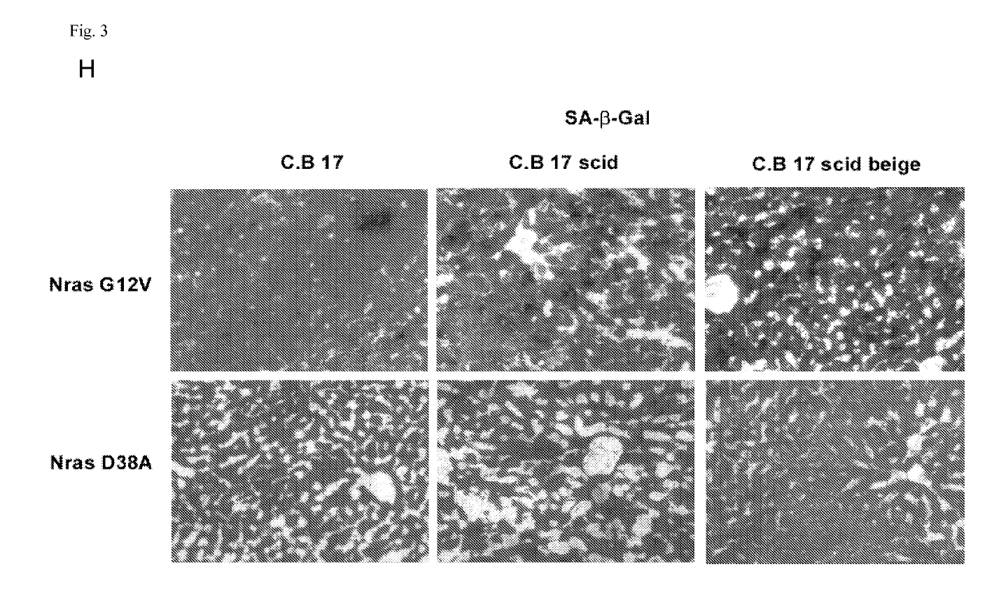

FIG. 3A-a schematically shows the experimental setup and controls to exclude an influence of the non-adaptive/innate immunity on the observed clearance of transformed cells, proving genetically an exclusive role for a specific cellular immune response being caused by the senescent state of premalignant liver cells,

FIGS. 3A-b, -c, -d, and -e show the quantification of immunostaining for Nras (b), p21 (c), p-Erk (d), and p16 (e) on liver sections of mice injected with the Nras G12V transposable element,

FIG. 3B shows micrographs of transfected liver cells at day 12 following the transformation with antibody staining against the antigen Nras,

FIG. 3C shows a graphical representation of the quantification of the antibody stains of the experiments over a period of 60 days,

FIG. 3D shows micrographs of liver cells with antibody staining against p21 at day 12 after the transformation,

FIG. 3E shows the quantification of the data depicted in FIG. 3d,

FIG. 3F shows micrographs of liver cells is staining against p-ERK at day 12 after transformation,

FIG. 3G shows the quantification of the analysis shown in FIG. 3f over the time course of 60 days for the presence of pERK,

FIG. 3H shows micrographs of liver cells with staining for SA-β-Gal at day 12 following transformation in the different mice strains transformed with each of the antigens

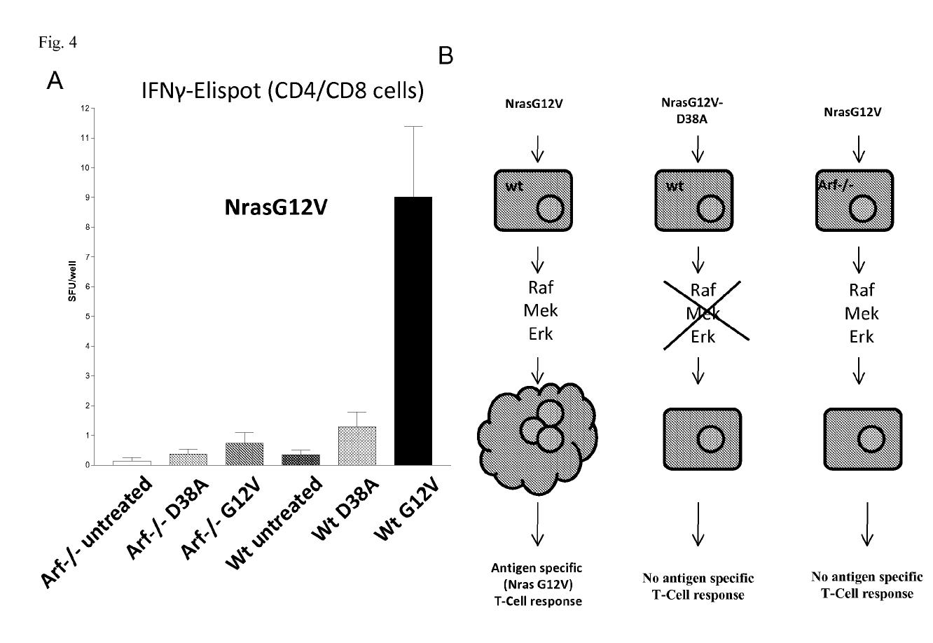

FIG. 4A shows the result of the IFN-γ-Elispot analysis,

FIG. 4B schematically summarizes the results deduced from the experimental evidence presented,

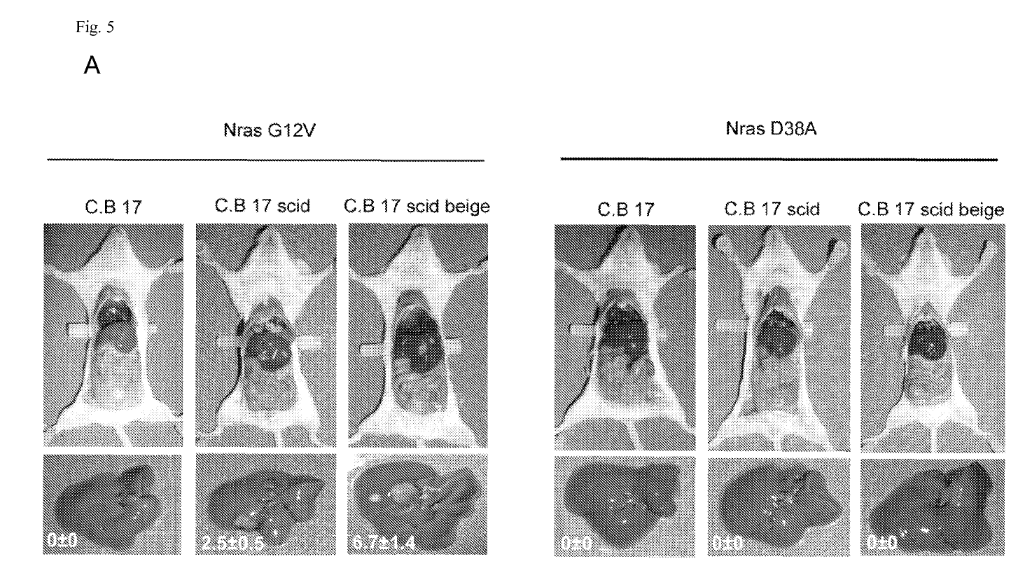

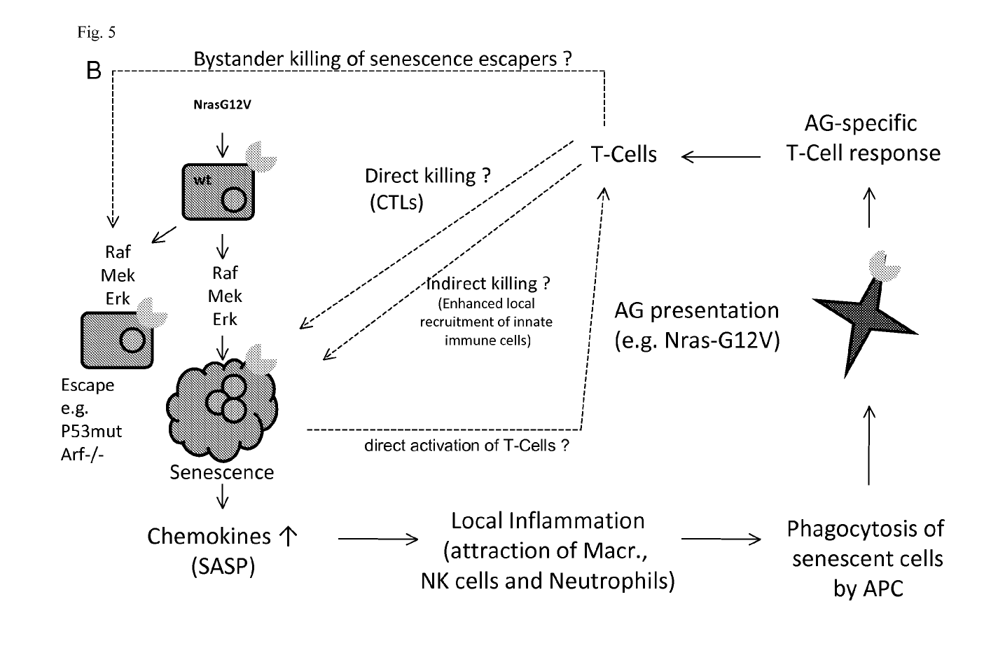

FIG. 5A shows tumour growth in the livers of mice, for mice with a fully competent immune system, for mice with an impaired immune system for essentially the same antigen characterising the malignant cells, with (Nras G12V) and without (Nras G12V D38A) induction of senescence in the malignant cells,

FIG. 5B schematically shows a first model reaction pathway which currently is deduced from the experimental evidence, which could be responsible for the observed generation of a specific immune response directed against malignant cells, which are e.g. characterized by expressing an antigen, if the premalignant cells are present in their senescent state, and the clearance of both senescent and non-senescent malignant cells by the specific immune response, which includes specific T-cells,

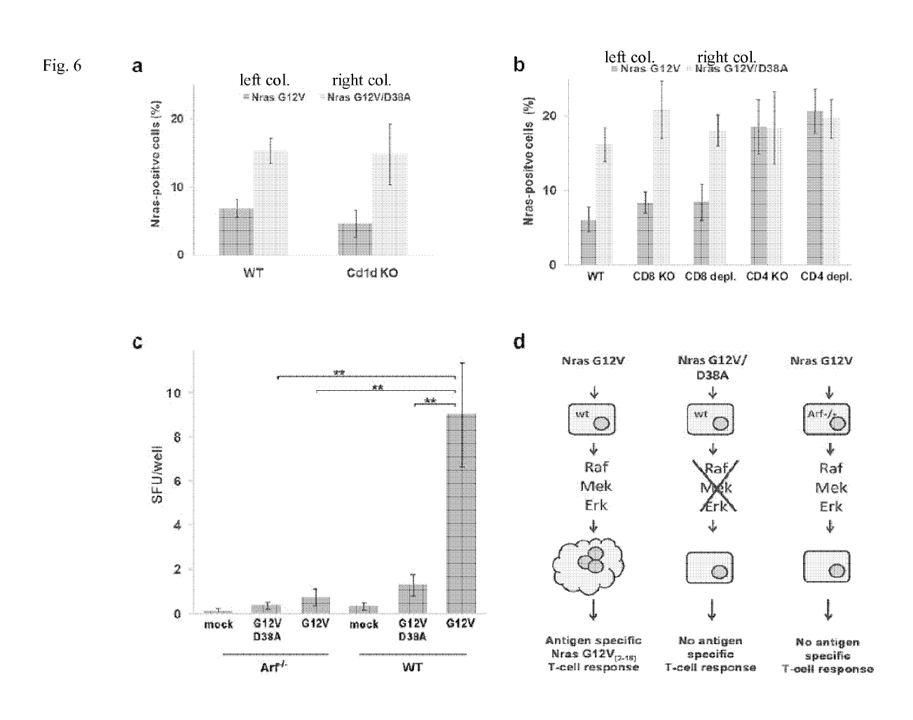

FIG. 6a shows the number of premalignant cells after presence of premalignant cells in their senescent state (Nras G12V) and in their non-senescent state (Nras G12V/D38A) in Cdld-knock-out mice,

FIG. 6b shows the number of premalignant cells after presence of premalignant cells in their senescent state (Nras G12V) and in their non-senescent state (Nras G12V/D38A) in CD8- and in CD4-negative mice, respectively,

FIG. 6c shows the result of an ELISPOT assay in mice after presence of premalignant cells in their senescent state (Nras G12V) and in their non-senescent state (Nras G12V/D38A) in wildtype and Arf-knock-out mice,

FIG. 6d schematically shows the dependency of the generation of the specific immune response on the presence of premalignant or malignant cells in their state of senescence, and

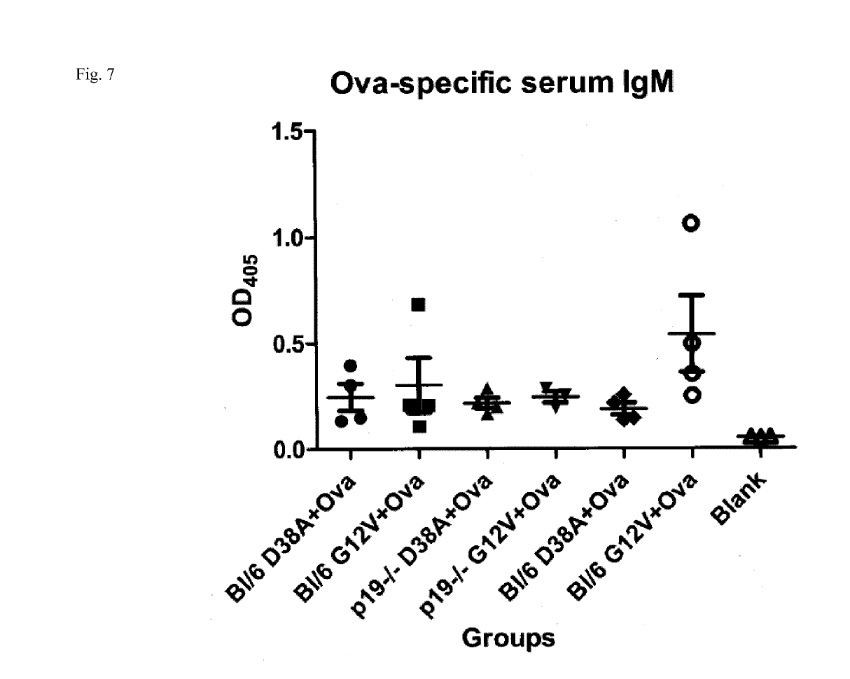

FIG. 7 shows ELISA results for a specific immune response directed against the model antigen HA due to the presence of senescent cells presenting this antigen.

### EXAMPLE

### Generation of a Specific Immune Response Directed Against an Antigen Expressed in Premalignant Cells in a Mammal

In this example, a mouse as a representative of a mammal is used to demonstrate the generation of a specific immune response including the generation of T-cells specifically directed against an antigen which is expressed in premalignant senescent cells. In this example, malignant cells are represented by the cells transformed to express the antigen. As shown in FIG. 1B, the livers of C57 BL/6 mice were stably transfected by delivery of nucleic acid constructs via hydrodynamic tailvein injection. In the nucleic acid constructs, which are schematically shown in FIG. 1A, transposons carrying the coding sequence for an oncogenic Nrasvariant, termed Nras G12V, or alternatively with a transposon carrying the coding sequence for the mutant of Nras G12V, termed Nras G12V D38A, which mutant carries an amino acid exchange in position 38, which affects signal transfer to MAP kinase. As a result, Nras G12V D38A is expressed and can be presented as an antigen, but does not by itself induce senescence. By contrast, Nras G12V induces senescence in the cells containing it. Both Nras G12V and its mutant D38A have very similar antigenic properties.

FIG. 1B shows micrographs of liver tissue 12 days after injection with the respective transposons, in staining with antibodies anti-Nras (α-Nras), anti-pErk (α-pERK), anti-p21 (aα-p21), and in senescence—associated β-galactosidase (SA-β-Gal) staining, it can be seen that cellular senescence is only induced by the oncogenic Nras G12V, whereas no senescence is induced by the mutant Nras G12V D38A.

The micrograph of FIG. 1C shows H- and E-staining of the liver tissue sections transfected with Nras G12V and of the liver tissue sections transfected with the comparative Nras G12V D38A.

FIG. 1D schematically shows the result that can be deduced from the experimental data, namely that it is only the antigen expressed in the exemplary premalignant cell, i.e. the tumour specific antigen Nras G12V which concurrently induces senescence, which leads to the induction of a specific immune response directed against the premalignant cells. Accordingly, it can be concluded that it is not only the presence of an antigen in malignant cells that strongly induces the specific T-cell response, because both Nras G12V and its mutant Nras G12V D38A are antigens. Accordingly, the induction of senescence in the malignant cell in accordance with the invention is responsible for the strong induction of a specific immune response, including a cellular immune response, e.g. inducing the generation of antigen-specific T-cells directed against malignant cells containing the antigen. This specific immune response can be directed against both senescent and non-senescent malignant as well as premalignant cells.

The micrographs of FIG. 1D show that the H- and E-staining, the α-Nras, α-pErk and α-p21 staining of liver sections transformed with Nras G12V, infiltrating immune cells can be seen in close proximity to senescent hepatocytes.

The micrographs of liver sections stained with α-Nras antibody at days 3, 12, 30 and 60 (D) for transfected cells containing Nras G12V and comparative Nras G12V D38A show that the number of Nras G12V positive cells decreases over time, whereas in the comparative experiments expressing Nras G12V D38A, the frequency of antigen—expressing hepatocytes is stable over time, indicating that the specific immune response is generated by the presence of senescent premalignant cells, which are represented in this example by senescent antigen expressing cells, and this immune response also leads to the elimination of non-senescent malignant cells.

A graphic representation of this observation is shown in FIG. 1G, wherein the number of antigen—positive cells, representing malignant cells, is efficiently reduced if the malignant cells in their senescent state were in contact with the immune cells, the senescent cells expressing an antigen specific for their malignancy (Nras G12V, lower curve, asymptotically approaching the baseline at day 60), whereas the antigen expressing cells which are not transformed to the senescence state (Nras D38A mutant, upper curve) does not induce an effective immune response that is capable of clearing malignant cells.

From the experimental animals transformed with transposons expressing Nras G12V or its mutant Nras G12V D38A (G12V D38A), on day 12 following transfection, portal lymph nodes (PLN) and livers of mice were harvested. Livers were perfused and digested to obtain single cell suspensions. Single cell suspensions were stained with antibodies against a number of cell surface markers to analyze and quantify immune cells as indicated in FIG. 2. As can be taken from FIG. 2A, the number of neutrophils is increased for transformants of Nras G12V in comparison to the mutant transformants D38A, well as a prominent increased, approximately by a factor of 17 of NK cells (NK1.1+CD11b high), as shown in FIG. 2B. Therefore, the innate immune reactions were shown to be highly activated in the mice injected with the oncogenic Nras G12V, i.e. in the presence of the antigen characterising a malignant cell in its senescent state, in comparison to the control cells (Nras G12V/D38A), i.e. without the induction of senescence in the malignant cells expressing the antigen with its one amino acid mutation.

The further analysis of PLN, the result of which is shown in FIG. 2C, reveals an increase (two to three fold) of dendritic cell (DC) populations for both antigens expressed. This result points to the maturation and migration of active DCs into the lymphoid centers, e.g. for antigen presentation (FIG. 2C).

As shown in FIG. 2D, the number of CD4+ T-cells present in the PLN were found to be elevated by a factor of approximately 1.3 for the cells transformed with G12V, with less pronounced differences in the numbers of CD8+ T-cells between G12V and mutant G12V/D38A expressing cells. These results show an increase in the number of CD4+ T-cells, and to a lesser extent of CD8+ T-cells in PLN based on the state of senescence of the premalignant cells characterized by expressing the antigen.

Recruitment and activation of CD4+ T-cells in the lymphoid centers would target these further to the local effector sites, e.g. to the liver, whether they will further differentiate, expand and provide activation signals, e.g. by means of the cytokines or via direct cell-to-cell contact, especially to CD 8+ T-cells, B-cells, and to innate immune cells, e.g. macrophages, natural killer cells (NK), and neutrophils. As can be seen from the results depicted in FIG. 2F, the recruitment of CD8+ T-cells in the liver has been found to be increased by a factor of about 1.7 in the numbers of CTLs in the G12V transformed group, i.e. when malignant cells characterized by the expression of antigen are present in their senescent state. As can be taken from FIGS. 2D and 2F, an increase of CD4+ T-helper cells in the livers of G12V—transformed animals is similar to the increase of T-helper cells seen in the draining PLN.

As seen from the results depicted in FIG. 2E, a pronounced recruitment of DCs is observed in the liver of animals expressing G12V, approximately by a factor of three in comparison to the control animals expressing the mutant G12V/D38A, which does not induce senescence in the malignant cells. This result also provides evidence for the increased attraction of immune cells to the effector site due to the state of senescence of the malignant cells, which are represented here by the antigen expressing cells.

A similar induction of a specific immune response, especially of specific T-cells directed against malignant cells, characterized by expressing an antigen as described for the exemplary antigen Nras G12V, could be obtained by inducing senescence in malignant or pre-malignant cells by applying a senescence inducing agent, e.g. by administration of a cytotoxic agent or by application of ionising irradiation at a dosage significantly below a cytotoxic dosage, e.g. at a dosage below the IC50, or by treatment with a PTEN-inhibitor, preferably VO-OHpic, or treatment with a MDM2-inhibitor, or by enforced re-expression of p53 or p14. Enforced re-expression of p53 or p14 could e.g. obtained by introduction of a DNA construct containing an expression cassette encoding p53 or p14, preferably in vitro using malignant cells obtained from the patient Accordingly, it could be shown that the activity of the Nras G12V to induce senescence in a transformed malignant cell, e.g. in the cell expressing the antigen Nras G12V, could also be obtained effectively be by inducing senescence in non-senescent malignant cells. The senescent cells, expressing Nras G12V as the antigen or the antigen characterizing the malignant cell (e.g. the antigen characterizing the autologous tumour cell) could induce a specific immune response, with both a cellular and a humoral immune response directed against the antigen.

A control experiment and the results depicted in FIG. 3 show that the increased specific immune response raised against malignant cells is induced by the presence of cells in their senescent state, by the functional immune system. In order to exclude effects of non-specific immune responses to the observed clearing of malignant cells, experimental animals, namely mice of the strain C.B 17 were transformed with transposons expressing Nras G12V as a model antigen characterising a malignant cell in its senescent state, and with a transposon encoding the mutant an Nras G12V D38A, which characterises the model malignant cells, but without induction of senescence in the malignant cells.

As shown in FIG. 3A-1, comparative experiments were performed to provide evidence that the specific immune response generated is a response to the antigen presented by the malignant cell in its senescent state, by excluding the possibility that there is a non-specific immune clearance of senescent pre-malignant cells. As described above, malignant and pre-malignant cells are represented in this example by hepatocytes transformed to express Nras G12V or Nras G12V D38A. As described above, nucleic acid constructs were injected into mice, namely into C.B 17 mice having a fully immuno-competent background, a mouse strain with a defective adaptive immunity, but having an intact innate immunity (C.B 17-SCID), and mice with a defective adapted and a defective innate immunity (C.B 17 SCID beige). Again, Nras G12V was used, which concurrent to providing an antigen that induces senescence in the cells, and the resultant cells are regarded as a model for senescent malignant cells, whereas the mutant protein Nras G12V D38A has essentially the same immunological properties, but does not induce senescence, and is therefore regarded as a comparative example.

As seen in FIG. 3B, staining of liver cells with antibody specific for Nras (Nras IHC) reveals clearance of malignant cells (Nras G12V transfected) in C.B 17 mice, whereas essentially no clearance was observed in the C.B 17 SCID and C.B 17 SCID beige immuno-defective mice at day 12. The control experiments with Nras G12V D38A show no clearance of transformed, i.e. malignant cells, again showing that it is the senescence of the malignant cells that a response and for inducing the specific immune response, especially the specific T-cell response to the antigen characterising the malignant cell.

The quantification of the analysis of clearance of transformed cells is shown in FIG. 3C, again demonstrating that without senescence of the malignant cells (Nras D38A), a significant number of transformed cells remains in the liver after 60 days following transformation, whereas with induction of senescence, an effective reduction the number of transformed cells, is achieved, but only in animals having a fully competent immune system, but not in animals having an impaired adaptive and/or impaired innate immune system.

The analytical results of FIG. 3D, giving micrographs of anti-p21-stained liver cells from the experimental animals at day 12 following transformation, show that clearance of p21-positive cells only occurs in CD 0.17 mice, but not in the immuno-defective strains C.B 17 SCID and C.B 17 SCID beige mice. Again, the control experiments with the antigen having essentially the same immunogenic properties (Nras G12V D38A) but without inducing senescence, essentially does not show clearance of malignant cells, irrespective of the genetic background of the immune system.

The quantification of the analysis of the presence of p21 as shown in FIG. 3E confirms the observations described for FIG. 3D. For the control transformation with Nras G12V D38A (Nras D38A), no p21-positive cells were found over the 60 days of analysis for all mouse strains.

FIG. 3F shows the analysis of anti-pERK antibody staining of liver tissue from the experimental animals at day 12 after transformation, showing clearance of pERK-positive cells over time in C.B 17 mice, whereas no clearance was observed in the immuno defective strains C.B 17 SCID and C.B 17 SCID beige.

Again, the control experiments using the antigen characterising the malignant cells, but without inducing senescence of the malignant cells (Nras G12V D38A) proves that no cell clearance regardless of the immune—background of the animals was observed without inducing senescence in the malignant cells.

The quantification of the analysis described for FIG. 3F is shown in FIG. 3G, and confirms the result.

FIG. 3H shows analytical stainings of liver cells from the experimental animal 12 days following transformation for SA-β-Gal. This analysis shows that less senescent cells are observed in the livers of immuno-competent C.B 17 mice than in the immuno-defective C.B 17 SCID or C.B 17 SCID beige mice, and essentially no senescent cells are found in the liver sections of the control, in which no senescence was induced.

This result proves that also non-senescent cells are cleared by the specific immune response, which is only induced in the presence of malignant cells in their senescent state, the specific immune response comprising specific T-cells directed against the malignant cells and its characterising antigen, respectively.

Using the identification of the cell populations, in which the TCR is able to recognise the model antigen characterising malignant cells, which is Nras Gl2V 2-17 peptide, the Nras G12V-specific immune responses were studied. Results show that the development of a specific adapted immune response occurs in the presence of senescent premalignant cells expressing the antigen, whereas liver cells expressing essentially the same antigen, but without induction of senescence do not as effectively induce the generation of a specific T-cell response.

In short, mice carrying the Arf −/− immune defect, which are therefore unable to produce a senescence response and antigen-specific immune cells, and wild-type C57 BL/6 mice were injected with Nras G12V D38A (control antigen, not inducing senescence), or an Nras G12V (model antigen characterising malignant cells, inducing senescence in malignant cells). 20 days post injection, murine spleenocytes were obtained, and cells were seeded in the wells of Elispot plates pre-coated with anti-IFN-γ antibodies. Spleenocytes were re-stimulated with Nras G12V 2-172 peptide, and IFN-gamma—secreting cells were detected following an incubation over 24 hours. T-cells having a TCR receptor and able to recognise the Nras G12V peptide after peptide re-stimulation undergo activation, resulting in the production of cytokines.

IFN-gamma is one of the most potent and primarily produced cytokines released by both CD4+ and CD8+ T-cells in response to presence of an antigen. The number of T-cells secreting IFN-gamma was the highest in mice injected with the oncogenic Nras G12V, whereas in the other experimental groups, these numbers were significantly lower. This shows that the senescence of malignant cells is essential for the induction of a specific immune response directed against the antigen characterizing the malignant cell, the specific immune response including CD8+ and CD4+ T-cells. Further, the numbers of IFN-gamma secreting cells were not elevated in the Arf−/− mice, in which the senescence response is blunted genetically, thus supporting the observation that without senescence of the premalignant cell, the high activity of the specific immune response is not generated, whereas it is the presence of the state of senescence in the malignant cells that induces the specific immune response directed against the malignant cells. The observed result is schematically depicted in FIG. 4B.

FIG. 5A shows exemplary experimental animals and their respective livers including any present tumour tissue at 8 months after injection with the respective antigen that is characterising the model malignant cell. It becomes clear that the invasive liver carcinomas were detected in immuno-defective C.B 17 SCID and C.B 17 SCID beige mice transformed with the oncogenic Nras G12V construct, whereas the immuno competent C.B 17 mice show complete clearance, i.e. no further invasive tumour growth of the liver. Further, the control mice treated with the mutant Nras G12V D38A irrespective of the genetic immune background show no tumour growth.

The working model that could be deduced from the results presented herein is schematically shown in FIG. 5B. Induction of senescence in the premalignant cell results in contacting of those cells with immune cells. As a result, immune cells having specificity for the malignant cells are generated in an effective way, comprising T-cells and B-cells producing antibody, having specificity each for the malignant cell. The specificity against the malignant cell can e.g. be the specificity for the antigen characterising the malignant cell, which specificity is exemplified by Nras G12V-specific clearing.

Currently, is assumed that the senescent state of malignant cells, e.g. due to the up-regulation of cytokines (SASP) which leads to e.g. local inflammation, and including the attraction of macrophage, NK-cells and neutrophils, subsequently to the phagocytosis of senescent cells by APC supports the antigen presentation by APC, which participate in the generation of antigen-specific T-cells.

The specificity of the immune cells generated in accordance with the invention by contact of immune cells with senescent malignant cells, especially of T-cells having specificity for the malignant cells, is directed both against the senescent malignant cells, and against the non-senescent malignant cells, e.g. a proliferating cells having escaped senescence or having escaped the innate immune response.

In a further experiment, it was found that the observed induction of an immune response that is specifically directed against malignant cells, which especially are tumour cells, is not an effect of NKT-cells against the malignant cells. In detail, Cd1d knock-out mice (lacking NKT-cells) were compared to syngenic wt-controls. Mice were treated by transduction of liver cells with a DNA construct containing an expression cassette encoding Nras G12V intrahepatically. 12 days after this intrahepatical delivery of Nras G12V, quantification of Nras-positive cells showed that the specific immune response against the malignant senescent cells expressing Nras G12V as the characterizing antigen was induced also in Cd1d-knock-out mice. This intact immune surveillance of premalignant senescent hepatocytes in absence of NKT-cells of these animals rules out NKT-cells as major effectors. Expression of the mutant Nras G12V D38A in Cd1d knock-out and wt-mice shows no induction of an immune response as seen for Nras G12V. Results are shown in FIG. 6a.

Further, CD8-knock-out mice and CD4-knockout mice as well as immuno-competent mice in which CD8+ and CD4+ cells were depleted using anti-CD8 and anti-CD4 antibodies were examined following transduction of liver cells with a DNA construct containing an expression cassette as described for the Cd1d-knock-out mice. As shown in the results of FIG. 6b, CD8-knock-out mice and CD8+-T-cell depleted mice showed about the efficacy of the specific immune response induced by senescent premalignant cells as seen in wt-mice. In CD4-knock-out mice and CD4+-T-cell depleted mice, essentially no effective immune response was observed. It is therefore assumed that the observed specific immune response that was caused by the senescent malignant cells (autologous senescent hepatocytes expressing the antigen Nras G12V) is a CD4+-T-cell dependent, antigen-specific immunity.

Using a mutant Nras-specific 15-mer peptide, an IFN-γ ELISPOT assay was performed on lymphocytes isolated from mice transduced with Nras G12V or Nras G12V D38A, respectively. The results are shown in FIG. 6c. In control mice (Nras G12V D38A) which did not contain senescent premalignant cells, only a few background positive lymphocytes were found, whereas in mice harbouring senescent malignant cells (Nras G12V), a significant increase in mutant ras-specific IFNγ-producing cells were found.

When repeating this experiment in p19Arf-knock-out mice, in which the senescence programme is genetically disabled, it was shown that the observed antigen-specific (ras) immune response is dependent on the presence of premalignant or malignant cells in their senescent state, and is not dependent on the ras-MAPK signalling cascade. Results are shown in FIG. 6d. Further, the intrahepatic delivery of Nras G12V into p19 Arf-knock-out mice did not trigger production of IFNγ. This shows that the induction of an antigen specific immune response according to the invention is dependent on the presence of premalignant or malignant cells in their senescent state.

As an example for any antigen expressed in a premalignant cell, influenza A-derived hemagglutinin (HA) was used. In short, wt mice were transduced with a transposable genetic element containing separate expression cassettes for Nras G12V and HA as schematically shown in FIG. 6e. ELISPOT assays that were performed on lymphocyte fractions from these mice using an MHCII-(I-E^{d}) specific HA-peptide indicated a strong, antigen-specific CD4+-T-cell dependent immune response induced by HA expressed by senescent hepatocytes. Results are shown in FIG. 6e. In another experiment, Ova was used as a model antigen. It could be shown that a cellular and humoral immune response specifically directed against Ova was induced, when Ova was presented by senescent cells in the experimental animal. The ELISA results are shown in FIG. 7 for co-expression of Nras G12V D38A with Ova (D38A+Ova), Nras G12V with Ova (G12V+Ova), each in the same genetic background of BI/6 mice, including the p19 (Arf)-knock-out mice. In FIG. 7, Blank indicates a sample from a non-transduced mouse. This result demonstrates that only when the antigen characterizing the malignant cell, exemplified here by Ova, is present in a senescent cell, the specific immune response is generated. In detail, no specific immune response is generated in the knock-out mice, which do not have the capacity for senescence, and no specific immune response is generated in presence of the D38A mutant ras, which does not induce senescence. Therefore, it is only the presence of the antigen in a senescent cell that generates the antigen-specific immune response, e.g. as shown here for the B-cell response.

1. Senescent pre-malignant or malignant cells for use as a medicament for inducing a specific immune response against senescent and non-senescent malignant cells in a treatment against malignant cells, wherein the pre-malignant or malignant senescent cells are generated by contact with a senescence inducing agent which is selected from cytostatic compounds, ionising radiation in a dosage which is essentially lower than a dosage inducing cell death, and an expression cassette causing overexpression of a tumour suppressor gene (e.g. p53) or of an oncogene, an MDM2 inhibiting drug, a PTEN inhibiting drug, and in that the dosage is sufficient for inducing senescence in at least some malignant cells.

2. Senescent pre-malignant or malignant cells according to claim 1, wherein the premalignant or malignant cells are tumour cells that are characterized by presentation of an antigen selected from a tumour-specific antigen, or the pre-malignant or malignant cells are virally or bacterially infected cells that are characterized by presentation of an antigen originating from a viral or bacterial infection.

3. Senescent pre-malignant or malignant cells according to claim 1, wherein the pre-malignant or malignant senescent cells which are contacted with a senescence inducing agent are autologous non-senescent pre-malignant or malignant isolated cells of the patient.

4. Senescent pre-malignant or malignant cells according to claim 1, wherein the pre-malignant or malignant senescent cells which are contacted with a senescence inducing agent are heterologous cells or autologous cells, which cells are genetically manipulated to contain a DNA construct expressing the antigen that is specific for the pre-malignant or malignant cell.

5. Senescent pre-malignant or malignant cells according to claim 1, wherein the specific immune response comprises T-cells directed against the malignant cells and/or B-cells producing antibody directed against the malignant cells.

6. Senescent pre-malignant or malignant cells according to claim 1, wherein a compound activating immune cells is present in combination with the senescence inducing agent or in combination with the senescent pre-malignant or malignant cells.

7. Senescent pre-malignant or malignant cells according to claim 1, wherein the senescent homologous malignant cell is formulated as a pharmaceutically acceptable formulation for implantation into the patient.

8. Immune cells for use as a medicament in a treatment against senescent and nonsenescent malignant cells, characterized in that the immune cells are primed for specificity against the pre-malignant and malignant cells by contact with senescent pre-malignant or malignant cells according to claim 1.

9. Immune cells according to claim 8, wherein the immune cells and the non-senescent malignant cells are autologous cells of one patient or heterologous cells from a different patient.

10. Immune cells according to claim 8, characterized in that the immune cells have acquired specificity for the malignant cell by contact of the immune cells with senescent malignant cells, and by integrating the immune cells into a pharmaceutically acceptable formulation for implantation into the patient.

11. Immune cells according to claim 8, characterized that the malignant cell is a tumour cell or a cell infected by a virus or a cell infected by a bacterium or a cell genetically manipulated to express a homologous or a heterologous antigen.

12. Process for producing senescent pre-malignant or malignant cells for use as a medicament in the treatment of malignant cells, the process comprising the steps of generating senescent malignant cells according to claim 1 by in vitro inducing senescence in autologous malignant cells, and formulating the senescent autologous malignant cells in a pharmaceutically acceptable formulation.

13. Process for producing antigen-specific immune cells for use as a medicament in the treatment of malignant cells, the process comprising the steps of generating senescent malignant cells according to claim 12 by in vitro inducing senescence in autologous malignant cells, in vitro contacting the senescent autologous malignant cells with autologous immune cells, and formulating the in a pharmaceutically acceptable formulation

14. Process according to claim 13, wherein following the step of in vitro contacting the senescent autologous or heterologous malignant cells with autologous immune cells, the autologous immune cells are separated from the senescent autologous malignant cells.

15. Process for producing antigen-specific immune cells, the process comprising the steps of generating senescent malignant cells according to claim 1 by transforming cells in an experimental animal with a nucleic acid construct encoding the antigen, inducing senescence in at least a fraction of the transformed cells within the experimental animal, followed by isolating spleen cells from the experimental animal, selecting immune cells having specificity for the antigen, and cultivating selected immune cells.

16. Process according to claim 15, characterized that the immune cells are B-cells, and a selected B-cell is fused with a tumour cell to generate a hybridoma, and cultivating the hybridoma for the production of antibody.

17. Pharmaceutical composition for use as a medicament in the treatment of malignant cells in a patient, the composition comprising in a pharmaceutically acceptable formulation a senescence inducing agent that is selected from cytostatic compounds and ionising radiation in a dosage which is essentially lower than a dosage inducing cell death, overexpression of a tumour suppressor gene or an oncogene and in that the dosage is sufficient for inducing senescence in at least some malignant cells.

18. Pharmaceutical composition according to claim 17, characterized that the pharmaceutical composition in combination with the senescence inducing agent comprises a compound activating immune cells.

19. Pharmaceutical composition for use in the medical treatment of malignant cells in a patient, the composition comprising in a pharmaceutically acceptable formulation for introduction into a patient an autologous immune cell of the patient, which immune cell is specific for the malignant cell by being contacted in vitro with a senescent malignant cell that is autologous to the patient.

20. Senescence inducing agent for use as a medicament in the generation of senescent premalignant or malignant cells according to claim 1, which is selected from cytostatic compounds, ionising radiation in a dosage which is essentially lower than a dosage inducing cell death, and an expression cassette causing overexpression of a tumour suppressor gene (e.g. p53) or of an oncogene, treatment with an MDM2 inhibitor, a PTEN inhibitor and in that the dosage is sufficient for inducing senescence in at least some malignant cells.


SEQUENCE LISTING (359 sequences)

SEQ ID NO: 1 - protein, 10 aa, Artificial Sequence
  SOURCE 1..10: /mol_type=“protein” /note=“tumour antigen for lung carcinoma” /organism=“Artificial Sequence”
FIASNGVKLV

SEQ ID NO: 2 - protein, 15 aa, Artificial Sequence
  SOURCE 1..15: /mol_type=“protein” /note=“tumour antigen for melanoma” /organism=“Artificial Sequence”
YSVYFNLPADTIYTN

SEQ ID NO: 3 - protein, 9 aa, Artificial Sequence
  SOURCE 1..9: /mol_type=“protein” /note=“tumour antigen for chronic myeloid leukemia” /organism=“Artificial Sequence”
SSKALQRPV

SEQ ID NO: 4 - protein, 9 aa, Artificial Sequence
  SOURCE 1..9: /mol_type=“protein” /note=“tumour antigen for chronic myeloid leukemia” /organism=“Artificial Sequence”
GFKQSSKAL

SEQ ID NO: 5 - protein, 17 aa, Artificial Sequence
  SOURCE 1..17: /mol_type=“protein” /note=“tumour antigen for chronic myeloid leukemia” /organism=“Artificial Sequence”
ATGFKQSSKALQRPVAS

SEQ ID NO: 6 - protein, 17 aa, Artificial Sequence
  SOURCE 1..17: /mol_type=“protein” /note=“tumour antigen for chronic myeloid leukemia” /organism=“Artificial Sequence”
ATGFKQSSKALQRPVAS

SEQ ID NO: 7 - protein, 29 aa, Artificial Sequence
  SOURCE 1..29: /mol_type=“protein” /note=“tumour antigen for melanoma” /organism=“Artificial Sequence”
EDLTVKIGDFGLATEKSRWSGSHQFEQLS

SEQ ID NO: 8 - protein, 9 aa, Artificial Sequence
  SOURCE 1..9: /mol_type=“protein” /note=“tumour antigen for colorectal, gastgric , and endometrialt umour” /organism=“Artificial Sequence”
FLIIWQNTM

SEQ ID NO: 9 - protein, 9 aa, Artificial Sequence
  SOURCE 1..9: /mol_type=“protein” /note=“tumour antigen for head and neck squamous cell carcinoma” /organism=“Artificial Sequence”
FPSDSWCYF

SEQ ID NO: 10 - protein, 9 aa, Artificial Sequence
  SOURCE 1..9: /mol_type=“protein” /note=“tumour antigen for melanoma” /organism=“Artificial Sequence”
SYLDSGIHF

SEQ ID NO: 11 - protein, 12 aa, Artificial Sequence
  SOURCE 1..12: /mol_type=“protein” /note=“tumour antigen for melanoma” /organism=“Artificial Sequence”
FSWAMDLDPKGA

SEQ ID NO: 12 - protein, 10 aa, Artificial Sequence
  SOURCE 1..10: /mol_type=“protein” /note=“tumour antigen for melanoma” /organism=“Artificial Sequence”
ACDPHSGHFV

SEQ ID NO: 13 - protein, 9 aa, Artificial Sequence
  SOURCE 1..9: /mol_type=“protein” /note=“tumour antigen for melanoma” /organism=“Artificial Sequence”
AVCPWTWLR

SEQ ID NO: 14 - protein, 14 aa, Artificial Sequence
  SOURCE 1..14: /mol_type=“protein” /note=“tumour antigen for colorectal carcinoma” /organism=“Artificial Sequence”
TLYQDDTLTLQAAG

SEQ ID NO: 15 - protein, 14 aa, Artificial Sequence
  SOURCE 1..14: /mol_type=“protein” /note=“tumour antigen for colorectal carcinoma” /organism=“Artificial Sequence”
TLYQDDTLTLQAAG

SEQ ID NO: 16 - protein, 16 aa, Artificial Sequence
  SOURCE 1..16: /mol_type=“protein” /note=“tumour antigen for myeloid leukemia” /organism=“Artificial Sequence”
TMKQICKKEIRRLHQY

SEQ ID NO: 17 - protein, 10 aa, Artificial Sequence
  SOURCE 1..10: /mol_type=“protein” /note=“tumour antigen for melanoma” /organism=“Artificial Sequence”
KILDAVVAQK

SEQ ID NO: 18 - protein, 9 aa, Artificial Sequence
  SOURCE 1..9: /mol_type=“protein” /note=“tumour antigen for lung squamous CC” /organism=“Artificial Sequence”
ETVSEQSNV

SEQ ID NO: 19 - protein, 9 aa, Artificial Sequence
  SOURCE 1..9: /mol_type=“protein” /note=“tumour antigen for acute lymphoblastic leukemia ” /organism=“Artificial Sequence”
RIAECILGM

SEQ ID NO: 20 - protein, 15 aa, Artificial Sequence
  SOURCE 1..15: /mol_type=“protein” /note=“tumour antigen for acute lymphoblastic leukemia ” /organism=“Artificial Sequence”
IGRIAECILGMNPSR

SEQ ID NO: 21 - protein, 15 aa, Artificial Sequence
  SOURCE 1..15: /mol_type=“protein” /note=“tumour antigen for acute lymphoblastic leukemia ” /organism=“Artificial Sequence”
IGRIAECILGMNPSR

SEQ ID NO: 22 - protein, 10 aa, Artificial Sequence
  SOURCE 1..10: /mol_type=“protein” /note=“tumour antigen for acute myelogenous leukemia ” /organism=“Artificial Sequence”
YVDFREYEYY

SEQ ID NO: 23 - protein, 14 aa, Artificial Sequence
  SOURCE 1..14: /mol_type=“protein” /note=“tumour antigen for melanoma” /organism=“Artificial Sequence”
MIFEKHGFRRTTPP

SEQ ID NO: 24 - protein, 10 aa, Artificial Sequence
  SOURCE 1..10: /mol_type=“protein” /note=“tumour antigen for melanoma” /organism=“Artificial Sequence”
TLDWLLQTPK

SEQ ID NO: 25 - protein, 9 aa, Artificial Sequence
  SOURCE 1..9: /mol_type=“protein” /note=“tumour antigen for melanoma” /organism=“Artificial Sequence”
WRRAPAPGA

SEQ ID NO: 26 - protein, 9 aa, Artificial Sequence
  SOURCE 1..9: /mol_type=“protein” /note=“tumour antigen for melanoma” /organism=“Artificial Sequence”
PVTWRRAPA

SEQ ID NO: 27 - protein, 10 aa, Artificial Sequence
  SOURCE 1..10: /mol_type=“protein” /note=“tumour antigen for renal cell carcinoma, for melanoma andr enal cell carcinoma ” /organism=“Artificial Sequence”
SLFEGIDIYT

SEQ ID NO: 28 - protein, 9 aa, Artificial Sequence
  SOURCE 1..9: /mol_type=“protein” /note=“tumour antigen for bladder tumour ” /organism=“Artificial Sequence”
AEPINIQTW

SEQ ID NO: 29 - protein, 11 aa, Artificial Sequence
  SOURCE 1..11: /mol_type=“protein” /note=“tumour antigen for melanoma” /organism=“Artificial Sequence”
FLEGNEVGKTY

SEQ ID NO: 30 - protein, 9 aa, Artificial Sequence
  SOURCE 1..9: /mol_type=“protein” /note=“tumour antigen for non-small cell lung carcinoma” /organism=“Artificial Sequence”
FLDEFMEGV

SEQ ID NO: 31 - protein, 9 aa, Artificial Sequence
  SOURCE 1..9: /mol_type=“protein” /note=“tumour antigen for melanoma” /organism=“Artificial Sequence”
EEKLIVVLF

SEQ ID NO: 32 - protein, 11 aa, Artificial Sequence
  SOURCE 1..11: /mol_type=“protein” /note=“tumour antigen for melanoma” /organism=“Artificial Sequence”
SELFRSGLDSY

SEQ ID NO: 33 - protein, 9 aa, Artificial Sequence
  SOURCE 1..9: /mol_type=“protein” /note=“tumour antigen for melanoma” /organism=“Artificial Sequence”
FRSGLDSYV

SEQ ID NO: 34 - protein, 9 aa, Artificial Sequence
  SOURCE 1..9: /mol_type=“protein” /note=“tumour antigen for melanoma” /organism=“Artificial Sequence”
EAFIQPITR

SEQ ID NO: 35 - protein, 11 aa, Artificial Sequence
  SOURCE 1..11: /mol_type=“protein” /note=“tumour antigen for melanoma” /organism=“Artificial Sequence”
RVIKNSIRLTL

SEQ ID NO: 36 - protein, 9 aa, Artificial Sequence
  SOURCE 1..9: /mol_type=“protein” /note=“tumour antigen for melanoma” /organism=“Artificial Sequence”
KINKNPKYK

SEQ ID NO: 37 - protein, 8 aa, Artificial Sequence
  SOURCE 1..8: /mol_type=“protein” /note=“tumour antigen for lung squamous cell carcinoma ” /organism=“Artificial Sequence”
QQITKTEV

SEQ ID NO: 38 - protein, 10 aa, Artificial Sequence
  SOURCE 1..10: /mol_type=“protein” /note=“tumour antigen for colorectal carcinoma ” /organism=“Artificial Sequence”
SLYKFSPFPL

SEQ ID NO: 39 - protein, 9 aa, Artificial Sequence
  SOURCE 1..9: /mol_type=“protein” /note=“tumour antigen for melanoma” /organism=“Artificial Sequence”
KELEGILLL

SEQ ID NO: 40 - protein, 9 aa, Artificial Sequence
  SOURCE 1..9: /mol_type=“protein” /note=“tumour antigen for head and neck squamous cell carcinoma” /organism=“Artificial Sequence”
VVPCEPPEV

SEQ ID NO: 41 - protein, 25 aa, Artificial Sequence
  SOURCE 1..25: /mol_type=“protein” /note=“tumour antigen for promyelocytic leukemia ” /organism=“Artificial Sequence”
NSNHVASGAGEAAIETQSSSSEEIV

SEQ ID NO: 42 - protein, 10 aa, Artificial Sequence
  SOURCE 1..10: /mol_type=“protein” /note=“tumour antigen for melanoma” /organism=“Artificial Sequence”
LLLDDLLVSI

SEQ ID NO: 43 - protein, 16 aa, Artificial Sequence
  SOURCE 1..16: /mol_type=“protein” /note=“tumour antigen for melanoma” /organism=“Artificial Sequence”
PYYFAAELPPRNLPEP

SEQ ID NO: 44 - protein, 9 aa, Artificial Sequence
  SOURCE 1..9: /mol_type=“protein” /note=“tumour antigen for pancreatic adenocarcinoma ” /organism=“Artificial Sequence”
VVVGAVGVG

SEQ ID NO: 45 - protein, 10 aa, Artificial Sequence
  SOURCE 1..10: /mol_type=“protein” /note=“tumour antigen for melanoma” /organism=“Artificial Sequence”
ILDTAGREEY

SEQ ID NO: 46 - protein, 9 aa, Artificial Sequence
  SOURCE 1..9: /mol_type=“protein” /note=“tumour antigen for melanoma” /organism=“Artificial Sequence”
RPHVPESAF

SEQ ID NO: 47 - protein, 9 aa, Artificial Sequence
  SOURCE 1..9: /mol_type=“protein” /note=“tumour antigen for melanoma” /organism=“Artificial Sequence”
KIFSEVTLK

SEQ ID NO: 48 - protein, 9 aa, Artificial Sequence
  SOURCE 1..9: /mol_type=“protein” /note=“tumour antigen for melanoma” /organism=“Artificial Sequence”
SHETVIIEL

SEQ ID NO: 49 - protein, 10 aa, Artificial Sequence
  SOURCE 1..10: /mol_type=“protein” /note=“tumour antigen for sarcoma” /organism=“Artificial Sequence”
QRPYGYDQIM

SEQ ID NO: 50 - protein, 9 aa, Artificial Sequence
  SOURCE 1..9: /mol_type=“protein” /note=“tumour antigen for colorectal carcinoma ” /organism=“Artificial Sequence”
RLSSCVPVA

SEQ ID NO: 51 - protein, 15 aa, Artificial Sequence
  SOURCE 1..15: /mol_type=“protein” /note=“tumour antigen for melanoma” /organism=“Artificial Sequence”
GELIGILNAAKVPAD

SEQ ID NO: 52 - protein, 9 aa, Artificial Sequence
  SOURCE 1..9: /mol_type=“protein” /note=“shared tumour antigen” /organism=“Artificial Sequence”
AARAVFLAL

SEQ ID NO: 53 - protein, 8 aa, Artificial Sequence
  SOURCE 1..8: /mol_type=“protein” /note=“shared tumour antigen” /organism=“Artificial Sequence”
YRPRPRRY

SEQ ID NO: 54 - protein, 9 aa, Artificial Sequence
  SOURCE 1..9: /mol_type=“protein” /note=“shared tumour antigen” /organism=“Artificial Sequence”
YYWPRPRRY

SEQ ID NO: 55 - protein, 10 aa, Artificial Sequence
  SOURCE 1..10: /mol_type=“protein” /note=“shared tumour antigen” /organism=“Artificial Sequence”
VLPDVFIRCV

SEQ ID NO: 56 - protein, 9 aa, Artificial Sequence
  SOURCE 1..9: /mol_type=“protein” /note=“shared tumour antigen” /organism=“Artificial Sequence”
MLAVISCAV

SEQ ID NO: 57 - protein, 9 aa, Artificial Sequence
  SOURCE 1..9: /mol_type=“protein” /note=“shared tumour antigen” /organism=“Artificial Sequence”
RQKRILVNL

SEQ ID NO: 58 - protein, 9 aa, Artificial Sequence
  SOURCE 1..9: /mol_type=“protein” /note=“shared tumour antigen” /organism=“Artificial Sequence”
NYNNFYRFL

SEQ ID NO: 59 - protein, 10 aa, Artificial Sequence
  SOURCE 1..10: /mol_type=“protein” /note=“shared tumour antigen” /organism=“Artificial Sequence”
EYSKECLKEF

SEQ ID NO: 60 - protein, 9 aa, Artificial Sequence
  SOURCE 1..9: /mol_type=“protein” /note=“shared tumour antigen” /organism=“Artificial Sequence”
EYLSLSDKI

SEQ ID NO: 61 - protein, 11 aa, Artificial Sequence
  SOURCE 1..11: /mol_type=“protein” /note=“shared tumour antigen” /organism=“Artificial Sequence”
MLMAQEALAFL

SEQ ID NO: 62 - protein, 9 aa, Artificial Sequence
  SOURCE 1..9: /mol_type=“protein” /note=“shared tumour antigen” /organism=“Artificial Sequence”
SLLMWITQC

SEQ ID NO: 63 - protein, 10 aa, Artificial Sequence
  SOURCE 1..10: /mol_type=“protein” /note=“shared tumour antigen” /organism=“Artificial Sequence”
LAAQERRVPR

SEQ ID NO: 64 - protein, 9 aa, Artificial Sequence
  SOURCE 1..9: /mol_type=“protein” /note=“shared tumour antigen” /organism=“Artificial Sequence”
ELVRRILSR

SEQ ID NO: 65 - protein, 9 aa, Artificial Sequence
  SOURCE 1..9: /mol_type=“protein” /note=“shared tumour antigen” /organism=“Artificial Sequence”
APRGVRMAV

SEQ ID NO: 66 - protein, 14 aa, Artificial Sequence
  SOURCE 1..14: /mol_type=“protein” /note=“shared tumour antigen” /organism=“Artificial Sequence”
SLLMWITQCFLPVF

SEQ ID NO: 67 - protein, 20 aa, Artificial Sequence
  SOURCE 1..20: /mol_type=“protein” /note=“shared tumour antigen” /organism=“Artificial Sequence”
QGAMLAAQERRVPRAAEVPR

SEQ ID NO: 68 - protein, 18 aa, Artificial Sequence
  SOURCE 1..18: /mol_type=“protein” /note=“shared tumour antigen” /organism=“Artificial Sequence”
AADHRQLQLSISSCLQQL

SEQ ID NO: 69 - protein, 22 aa, Artificial Sequence
  SOURCE 1..22: /mol_type=“protein” /note=“shared tumour antigen” /organism=“Artificial Sequence”
CLSRRPWKRSWSAGSCPGMPHL

SEQ ID NO: 70 - protein, 22 aa, Artificial Sequence
  SOURCE 1..22: /mol_type=“protein” /note=“shared tumour antigen” /organism=“Artificial Sequence”
CLSRRPWKRSWSAGSCPGMPHL

SEQ ID NO: 71 - protein, 13 aa, Artificial Sequence
  SOURCE 1..13: /mol_type=“protein” /note=“shared tumour antigen” /organism=“Artificial Sequence”
ILSRDAAPLPRPG

SEQ ID NO: 72 - protein, 14 aa, Artificial Sequence
  SOURCE 1..14: /mol_type=“protein” /note=“shared tumour antigen” /organism=“Artificial Sequence”
AGATGGRGPRGAGA

SEQ ID NO: 73 - protein, 9 aa, Artificial Sequence
  SOURCE 1..9: /mol_type=“protein” /note=“shared tumour antigen” /organism=“Artificial Sequence”
EADPTGHSY

SEQ ID NO: 74 - protein, 9 aa, Artificial Sequence
  SOURCE 1..9: /mol_type=“protein” /note=“shared tumour antigen” /organism=“Artificial Sequence”
KVLEYVIKV

SEQ ID NO: 75 - protein, 9 aa, Artificial Sequence
  SOURCE 1..9: /mol_type=“protein” /note=“shared tumour antigen” /organism=“Artificial Sequence”
SLFRAVITK

SEQ ID NO: 76 - protein, 10 aa, Artificial Sequence
  SOURCE 1..10: /mol_type=“protein” /note=“shared tumour antigen” /organism=“Artificial Sequence”
EVYDGREHSA

SEQ ID NO: 77 - protein, 9 aa, Artificial Sequence
  SOURCE 1..9: /mol_type=“protein” /note=“shared tumour antigen” /organism=“Artificial Sequence”
RVRFFFPSL

SEQ ID NO: 78 - protein, 9 aa, Artificial Sequence
  SOURCE 1..9: /mol_type=“protein” /note=“shared tumour antigen” /organism=“Artificial Sequence”
EADPTGHSY

SEQ ID NO: 79 - protein, 10 aa, Artificial Sequence
  SOURCE 1..10: /mol_type=“protein” /note=“shared tumour antigen” /organism=“Artificial Sequence”
REPVTKAEML

SEQ ID NO: 80 - protein, 9 aa, Artificial Sequence
  SOURCE 1..9: /mol_type=“protein” /note=“shared tumour antigen” /organism=“Artificial Sequence”
DPARYEFLW

SEQ ID NO: 81 - protein, 11 aa, Artificial Sequence
  SOURCE 1..11: /mol_type=“protein” /note=“shared tumour antigen” /organism=“Artificial Sequence”
ITKKVADLVGF

SEQ ID NO: 82 - protein, 9 aa, Artificial Sequence
  SOURCE 1..9: /mol_type=“protein” /note=“shared tumour antigen” /organism=“Artificial Sequence”
SAFPTTINF

SEQ ID NO: 83 - protein, 9 aa, Artificial Sequence
  SOURCE 1..9: /mol_type=“protein” /note=“shared tumour antigen” /organism=“Artificial Sequence”
SAYGEPRKL

SEQ ID NO: 84 - protein, 9 aa, Artificial Sequence
  SOURCE 1..9: /mol_type=“protein” /note=“shared tumour antigen” /organism=“Artificial Sequence”
SAYGEPRKL

SEQ ID NO: 85 - protein, 15 aa, Artificial Sequence
  SOURCE 1..15: /mol_type=“protein” /note=“shared tumour antigen” /organism=“Artificial Sequence”
TSCILESLFRAVITK

SEQ ID NO: 86 - protein, 15 aa, Artificial Sequence
  SOURCE 1..15: /mol_type=“protein” /note=“shared tumour antigen” /organism=“Artificial Sequence”
PRALAETSYVKVLEY

SEQ ID NO: 87 - protein, 16 aa, Artificial Sequence
  SOURCE 1..16: /mol_type=“protein” /note=“shared tumour antigen” /organism=“Artificial Sequence”
FLLLKYRAREPVTKAE

SEQ ID NO: 88 - protein, 12 aa, Artificial Sequence
  SOURCE 1..12: /mol_type=“protein” /note=“shared tumour antigen” /organism=“Artificial Sequence”
EYVIKVSARVRF

SEQ ID NO: 89 - protein, 10 aa, Artificial Sequence
  SOURCE 1..10: /mol_type=“protein” /note=“shared tumour antigen” /organism=“Artificial Sequence”
YLQLVFGIEV

SEQ ID NO: 90 - protein, 9 aa, Artificial Sequence
  SOURCE 1..9: /mol_type=“protein” /note=“shared tumour antigen” /organism=“Artificial Sequence”
EYLQLVFGI

SEQ ID NO: 91 - protein, 10 aa, Artificial Sequence
  SOURCE 1..10: /mol_type=“protein” /note=“shared tumour antigen” /organism=“Artificial Sequence”
REPVTKAEML

SEQ ID NO: 92 - protein, 9 aa, Artificial Sequence
  SOURCE 1..9: /mol_type=“protein” /note=“shared tumour antigen” /organism=“Artificial Sequence”
EGDCAPEEK

SEQ ID NO: 93 - protein, 14 aa, Artificial Sequence
  SOURCE 1..14: /mol_type=“protein” /note=“shared tumour antigen” /organism=“Artificial Sequence”
LLKYRAREPVTKAE

SEQ ID NO: 94 - protein, 9 aa, Artificial Sequence
  SOURCE 1..9: /mol_type=“protein” /note=“shared tumour antigen” /organism=“Artificial Sequence”
EVDPIGHLY

SEQ ID NO: 95 - protein, 9 aa, Artificial Sequence
  SOURCE 1..9: /mol_type=“protein” /note=“shared tumour antigen” /organism=“Artificial Sequence”
FLWGPRALV

SEQ ID NO: 96 - protein, 9 aa, Artificial Sequence
  SOURCE 1..9: /mol_type=“protein” /note=“shared tumour antigen” /organism=“Artificial Sequence”
KVAELVHFL

SEQ ID NO: 97 - protein, 9 aa, Artificial Sequence
  SOURCE 1..9: /mol_type=“protein” /note=“shared tumour antigen” /organism=“Artificial Sequence”
TFPDLESEF

SEQ ID NO: 98 - protein, 9 aa, Artificial Sequence
  SOURCE 1..9: /mol_type=“protein” /note=“shared tumour antigen” /organism=“Artificial Sequence”
VAELVHFLL

SEQ ID NO: 99 - protein, 10 aa, Artificial Sequence
  SOURCE 1..10: /mol_type=“protein” /note=“shared tumour antigen” /organism=“Artificial Sequence”
MEVDPIGHLY

SEQ ID NO: 100 - protein, 9 aa, Artificial Sequence
  SOURCE 1..9: /mol_type=“protein” /note=“shared tumour antigen” /organism=“Artificial Sequence”
EVDPIGHLY

SEQ ID NO: 101 - protein, 10 aa, Artificial Sequence
  SOURCE 1..10: /mol_type=“protein” /note=“shared tumour antigen” /organism=“Artificial Sequence”
REPVTKAEML

SEQ ID NO: 102 - protein, 9 aa, Artificial Sequence
  SOURCE 1..9: /mol_type=“protein” /note=“shared tumour antigen” /organism=“Artificial Sequence”
AELVHFLLL

SEQ ID NO: 103 - protein, 10 aa, Artificial Sequence
  SOURCE 1..10: /mol_type=“protein” /note=“shared tumour antigen” /organism=“Artificial Sequence”
MEVDPIGHLY

SEQ ID NO: 104 - protein, 9 aa, Artificial Sequence
  SOURCE 1..9: /mol_type=“protein” /note=“shared tumour antigen” /organism=“Artificial Sequence”
WQYFFPVIF

SEQ ID NO: 105 - protein, 9 aa, Artificial Sequence
  SOURCE 1..9: /mol_type=“protein” /note=“shared tumour antigen” /organism=“Artificial Sequence”
EGDCAPEEK

SEQ ID NO: 106 - protein, 16 aa, Artificial Sequence
  SOURCE 1..16: /mol_type=“protein” /note=“shared tumour antigen” /organism=“Artificial Sequence”
KKLLTQHFVQENYLEY

SEQ ID NO: 107 - protein, 16 aa, Artificial Sequence
  SOURCE 1..16: /mol_type=“protein” /note=“shared tumour antigen” /organism=“Artificial Sequence”
KKLLTQHFVQENYLEY

SEQ ID NO: 108 - protein, 16 aa, Artificial Sequence
  SOURCE 1..16: /mol_type=“protein” /note=“shared tumour antigen” /organism=“Artificial Sequence”
ACYEFLWGPRALVETS

SEQ ID NO: 109 - protein, 12 aa, Artificial Sequence
  SOURCE 1..12: /mol_type=“protein” /note=“shared tumour antigen” /organism=“Artificial Sequence”
VIFSKASSSLQL

SEQ ID NO: 110 - protein, 12 aa, Artificial Sequence
  SOURCE 1..12: /mol_type=“protein” /note=“shared tumour antigen” /organism=“Artificial Sequence”
VIFSKASSSLQL

SEQ ID NO: 111 - protein, 15 aa, Artificial Sequence
  SOURCE 1..15: /mol_type=“protein” /note=“shared tumour antigen” /organism=“Artificial Sequence”
GDNQIMPKAGLLIIV

SEQ ID NO: 112 - protein, 15 aa, Artificial Sequence
  SOURCE 1..15: /mol_type=“protein” /note=“shared tumour antigen” /organism=“Artificial Sequence”
TSYVKVLHHMVKISG

SEQ ID NO: 113 - protein, 16 aa, Artificial Sequence
  SOURCE 1..16: /mol_type=“protein” /note=“shared tumour antigen” /organism=“Artificial Sequence”
RKVAELVHFLLLKYRA

SEQ ID NO: 114 - protein, 16 aa, Artificial Sequence
  SOURCE 1..16: /mol_type=“protein” /note=“shared tumour antigen” /organism=“Artificial Sequence”
FLLLKYRAREPVTKAE

SEQ ID NO: 115 - protein, 9 aa, Artificial Sequence
  SOURCE 1..9: /mol_type=“protein” /note=“shared tumour antigen” /organism=“Artificial Sequence”
EVDPASNTY

SEQ ID NO: 116 - protein, 10 aa, Artificial Sequence
  SOURCE 1..10: /mol_type=“protein” /note=“shared tumour antigen” /organism=“Artificial Sequence”
GVYDGREHTV

SEQ ID NO: 117 - protein, 9 aa, Artificial Sequence
  SOURCE 1..9: /mol_type=“protein” /note=“shared tumour antigen” /organism=“Artificial Sequence”
NYKRCFPVI

SEQ ID NO: 118 - protein, 8 aa, Artificial Sequence
  SOURCE 1..8: /mol_type=“protein” /note=“shared tumour antigen” /organism=“Artificial Sequence”
SESLKMIF

SEQ ID NO: 119 - protein, 9 aa, Artificial Sequence
  SOURCE 1..9: /mol_type=“protein” /note=“shared tumour antigen” /organism=“Artificial Sequence”
MVKISGGPR

SEQ ID NO: 120 - protein, 9 aa, Artificial Sequence
  SOURCE 1..9: /mol_type=“protein” /note=“shared tumour antigen” /organism=“Artificial Sequence”
EVDPIGHVY

SEQ ID NO: 121 - protein, 10 aa, Artificial Sequence
  SOURCE 1..10: /mol_type=“protein” /note=“shared tumour antigen” /organism=“Artificial Sequence”
REPVTKAEML

SEQ ID NO: 122 - protein, 9 aa, Artificial Sequence
  SOURCE 1..9: /mol_type=“protein” /note=“shared tumour antigen” /organism=“Artificial Sequence”
EGDCAPEEK

SEQ ID NO: 123 - protein, 9 aa, Artificial Sequence
  SOURCE 1..9: /mol_type=“protein” /note=“shared tumour antigen” /organism=“Artificial Sequence”
ISGGPRISY

SEQ ID NO: 124 - protein, 14 aa, Artificial Sequence
  SOURCE 1..14: /mol_type=“protein” /note=“shared tumour antigen” /organism=“Artificial Sequence”
LLKYRAREPVTKAE

SEQ ID NO: 125 - protein, 9 aa, Artificial Sequence
  SOURCE 1..9: /mol_type=“protein” /note=“shared tumour antigen” /organism=“Artificial Sequence”
ALSVMGVYV

SEQ ID NO: 126 - protein, 9 aa, Artificial Sequence
  SOURCE 1..9: /mol_type=“protein” /note=“shared tumour antigen” /organism=“Artificial Sequence”
GLYDGMEHL

SEQ ID NO: 127 - protein, 9 aa, Artificial Sequence
  SOURCE 1..9: /mol_type=“protein” /note=“shared tumour antigen” /organism=“Artificial Sequence”
DPARYEFLW

SEQ ID NO: 128 - protein, 9 aa, Artificial Sequence
  SOURCE 1..9: /mol_type=“protein” /note=“shared tumour antigen” /organism=“Artificial Sequence”
FLWGPRALV

SEQ ID NO: 129 - protein, 9 aa, Artificial Sequence
  SOURCE 1..9: /mol_type=“protein” /note=“shared tumour antigen” /organism=“Artificial Sequence”
VRIGHLYIL

SEQ ID NO: 130 - protein, 9 aa, Artificial Sequence
  SOURCE 1..9: /mol_type=“protein” /note=“shared tumour antigen” /organism=“Artificial Sequence”
EGDCAPEEK

SEQ ID NO: 131 - protein, 15 aa, Artificial Sequence
  SOURCE 1..15: /mol_type=“protein” /note=“shared tumour antigen” /organism=“Artificial Sequence”
REPFTKAEMLGSVIR

SEQ ID NO: 132 - protein, 14 aa, Artificial Sequence
  SOURCE 1..14: /mol_type=“protein” /note=“shared tumour antigen” /organism=“Artificial Sequence”
AELVHFLLLKYRAR

SEQ ID NO: 133 - protein, 10 aa, Artificial Sequence
  SOURCE 1..10: /mol_type=“protein” /note=“shared tumour antigen” /organism=“Artificial Sequence”
LLFGLALIEV

SEQ ID NO: 134 - protein, 9 aa, Artificial Sequence
  SOURCE 1..9: /mol_type=“protein” /note=“shared tumour antigen” /organism=“Artificial Sequence”
ALKDVEERV

SEQ ID NO: 135 - protein, 9 aa, Artificial Sequence
  SOURCE 1..9: /mol_type=“protein” /note=“shared tumour antigen” /organism=“Artificial Sequence”
SESIKKKVL

SEQ ID NO: 136 - protein, 20 aa, Artificial Sequence
  SOURCE 1..20: /mol_type=“protein” /note=“shared tumour antigen” /organism=“Artificial Sequence”
PDTRPAPGSTAPPAHGVTSA

SEQ ID NO: 137 - protein, 9 aa, Artificial Sequence
  SOURCE 1..9: /mol_type=“protein” /note=“shared tumour antigen” /organism=“Artificial Sequence”
QGQHFLQKV

SEQ ID NO: 138 - protein, 9 aa, Artificial Sequence
  SOURCE 1..9: /mol_type=“protein” /note=“shared tumour antigen” /organism=“Artificial Sequence”
SLLMWITQC

SEQ ID NO: 139 - protein, 11 aa, Artificial Sequence
  SOURCE 1..11: /mol_type=“protein” /note=“shared tumour antigen” /organism=“Artificial Sequence”
MLMAQEALAFL

SEQ ID NO: 140 - protein, 10 aa, Artificial Sequence
  SOURCE 1..10: /mol_type=“protein” /note=“shared tumour antigen” /organism=“Artificial Sequence”
ASGPGGGAPR

SEQ ID NO: 141 - protein, 10 aa, Artificial Sequence
  SOURCE 1..10: /mol_type=“protein” /note=“shared tumour antigen” /organism=“Artificial Sequence”
LAAQERRVPR

SEQ ID NO: 142 - protein, 10 aa, Artificial Sequence
  SOURCE 1..10: /mol_type=“protein” /note=“shared tumour antigen” /organism=“Artificial Sequence”
TVSGNILTIR

SEQ ID NO: 143 - protein, 13 aa, Artificial Sequence
  SOURCE 1..13: /mol_type=“protein” /note=“shared tumour antigen” /organism=“Artificial Sequence”
APRGPHGGAASGL

SEQ ID NO: 144 - protein, 9 aa, Artificial Sequence
  SOURCE 1..9: /mol_type=“protein” /note=“shared tumour antigen” /organism=“Artificial Sequence”
MPFATPMEA

SEQ ID NO: 145 - protein, 12 aa, Artificial Sequence
  SOURCE 1..12: /mol_type=“protein” /note=“shared tumour antigen” /organism=“Artificial Sequence”
KEFTVSGNILTI

SEQ ID NO: 146 - protein, 9 aa, Artificial Sequence
  SOURCE 1..9: /mol_type=“protein” /note=“shared tumour antigen” /organism=“Artificial Sequence”
MPFATPMEA

SEQ ID NO: 147 - protein, 9 aa, Artificial Sequence
  SOURCE 1..9: /mol_type=“protein” /note=“shared tumour antigen” /organism=“Artificial Sequence”
LAMPFATPM

SEQ ID NO: 148 - protein, 9 aa, Artificial Sequence
  SOURCE 1..9: /mol_type=“protein” /note=“shared tumour antigen” /organism=“Artificial Sequence”
ARGPESRLL

SEQ ID NO: 149 - protein, 14 aa, Artificial Sequence
  SOURCE 1..14: /mol_type=“protein” /note=“shared tumour antigen” /organism=“Artificial Sequence”
SLLMWITQCFLPVF

SEQ ID NO: 150 - protein, 25 aa, Artificial Sequence
  SOURCE 1..25: /mol_type=“protein” /note=“shared tumour antigen” /organism=“Artificial Sequence”
LLEFYLAMPFATPMEAELARRSLAQ

SEQ ID NO: 151 - protein, 25 aa, Artificial Sequence
  SOURCE 1..25: /mol_type=“protein” /note=“shared tumour antigen” /organism=“Artificial Sequence”
LLEFYLAMPFATPMEAELARRSLAQ

SEQ ID NO: 152 - protein, 12 aa, Artificial Sequence
  SOURCE 1..12: /mol_type=“protein” /note=“shared tumour antigen” /organism=“Artificial Sequence”
EFYLAMPFATPM

SEQ ID NO: 153 - protein, 12 aa, Artificial Sequence
  SOURCE 1..12: /mol_type=“protein” /note=“shared tumour antigen” /organism=“Artificial Sequence”
RLLEFYLAMPFA

SEQ ID NO: 154 - protein, 20 aa, Artificial Sequence
  SOURCE 1..20: /mol_type=“protein” /note=“shared tumour antigen” /organism=“Artificial Sequence”
QGAMLAAQERRVPRAAEVPR

SEQ ID NO: 155 - protein, 20 aa, Artificial Sequence
  SOURCE 1..20: /mol_type=“protein” /note=“shared tumour antigen” /organism=“Artificial Sequence”
PGVLLKEFTVSGNILTIRLT

SEQ ID NO: 156 - protein, 10 aa, Artificial Sequence
  SOURCE 1..10: /mol_type=“protein” /note=“shared tumour antigen” /organism=“Artificial Sequence”
VLLKEFTVSG

SEQ ID NO: 157 - protein, 18 aa, Artificial Sequence
  SOURCE 1..18: /mol_type=“protein” /note=“shared tumour antigen” /organism=“Artificial Sequence”
AADHRQLQLSISSCLQQL

SEQ ID NO: 158 - protein, 25 aa, Artificial Sequence
  SOURCE 1..25: /mol_type=“protein” /note=“shared tumour antigen” /organism=“Artificial Sequence”
LLEFYLAMPFATPMEAELARRSLAQ

SEQ ID NO: 159 - protein, 15 aa, Artificial Sequence
  SOURCE 1..15: /mol_type=“protein” /note=“shared tumour antigen” /organism=“Artificial Sequence”
LKEFTVSGNILTIRL

SEQ ID NO: 160 - protein, 25 aa, Artificial Sequence
  SOURCE 1..25: /mol_type=“protein” /note=“shared tumour antigen” /organism=“Artificial Sequence”
PGVLLKEFTVSGNILTIRLTAADHR

SEQ ID NO: 161 - protein, 25 aa, Artificial Sequence
  SOURCE 1..25: /mol_type=“protein” /note=“shared tumour antigen” /organism=“Artificial Sequence”
LLEFYLAMPFATPMEAELARRSLAQ

SEQ ID NO: 162 - protein, 14 aa, Artificial Sequence
  SOURCE 1..14: /mol_type=“protein” /note=“shared tumour antigen” /organism=“Artificial Sequence”
AGATGGRGPRGAGA

SEQ ID NO: 163 - protein, 9 aa, Artificial Sequence
  SOURCE 1..9: /mol_type=“protein” /note=“shared tumour antigen” /organism=“Artificial Sequence”
LYATVIHDI

SEQ ID NO: 164 - protein, 9 aa, Artificial Sequence
  SOURCE 1..9: /mol_type=“protein” /note=“shared tumour antigen” /organism=“Artificial Sequence”
ILDSSEEDK

SEQ ID NO: 165 - protein, 9 aa, Artificial Sequence
  SOURCE 1..9: /mol_type=“protein” /note=“shared tumour antigen” /organism=“Artificial Sequence”
KASEKIFYV

SEQ ID NO: 166 - protein, 16 aa, Artificial Sequence
  SOURCE 1..16: /mol_type=“protein” /note=“shared tumour antigen” /organism=“Artificial Sequence”
EKIQKAFDDIAKYFSK

SEQ ID NO: 167 - protein, 18 aa, Artificial Sequence
  SOURCE 1..18: /mol_type=“protein” /note=“shared tumour antigen” /organism=“Artificial Sequence”
WEKMKASEKIFYVYMKRK

SEQ ID NO: 168 - protein, 15 aa, Artificial Sequence
  SOURCE 1..15: /mol_type=“protein” /note=“shared tumour antigen” /organism=“Artificial Sequence”
KIFYVYMKRKYEAMT

SEQ ID NO: 169 - protein, 14 aa, Artificial Sequence
  SOURCE 1..14: /mol_type=“protein” /note=“shared tumour antigen” /organism=“Artificial Sequence”
KIFYVYMKRKYEAM

SEQ ID NO: 170 - protein, 20 aa, Artificial Sequence
  SOURCE 1..20: /mol_type=“protein” /note=“shared tumour antigen” /organism=“Artificial Sequence”
INKTSGPKRGKHAWTHRLRE

SEQ ID NO: 171 - protein, 20 aa, Artificial Sequence
  SOURCE 1..20: /mol_type=“protein” /note=“shared tumour antigen” /organism=“Artificial Sequence”
YFSKKEWEKMKSSEKIVYVY

SEQ ID NO: 172 - protein, 20 aa, Artificial Sequence
  SOURCE 1..20: /mol_type=“protein” /note=“shared tumour antigen” /organism=“Artificial Sequence”
MKLNYEVMTKLGFKVTLPPF

SEQ ID NO: 173 - protein, 20 aa, Artificial Sequence
  SOURCE 1..20: /mol_type=“protein” /note=“shared tumour antigen” /organism=“Artificial Sequence”
KHAWTHRLRERKQLVVYEEI

SEQ ID NO: 174 - protein, 20 aa, Artificial Sequence
  SOURCE 1..20: /mol_type=“protein” /note=“shared tumour antigen” /organism=“Artificial Sequence”
LGFKVTLPPFMRSKRAADFH

SEQ ID NO: 175 - protein, 20 aa, Artificial Sequence
  SOURCE 1..20: /mol_type=“protein” /note=“shared tumour antigen” /organism=“Artificial Sequence”
KSSEKIVYVYMKLNYEVMTK

SEQ ID NO: 176 - protein, 20 aa, Artificial Sequence
  SOURCE 1..20: /mol_type=“protein” /note=“shared tumour antigen” /organism=“Artificial Sequence”
KHAWTHRLRERKQLVVYEEI

SEQ ID NO: 177 - protein, 9 aa, Artificial Sequence
  SOURCE 1..9: /mol_type=“protein” /note=“shared tumour antigen” /organism=“Artificial Sequence”
SLGWLFLLL

SEQ ID NO: 178 - protein, 9 aa, Artificial Sequence
  SOURCE 1..9: /mol_type=“protein” /note=“shared tumour antigen” /organism=“Artificial Sequence”
LSRLSNRLL

SEQ ID NO: 179 - protein, 9 aa, Artificial Sequence
  SOURCE 1..9: /mol_type=“protein” /note=“shared tumour antigen” /organism=“Artificial Sequence”
LSRLSNRLL

SEQ ID NO: 180 - protein, 15 aa, Artificial Sequence
  SOURCE 1..15: /mol_type=“protein” /note=“shared tumour antigen” /organism=“Artificial Sequence”
CEFHACWPAFTVLGE

SEQ ID NO: 181 - protein, 15 aa, Artificial Sequence
  SOURCE 1..15: /mol_type=“protein” /note=“shared tumour antigen” /organism=“Artificial Sequence”
CEFHACWPAFTVLGE

SEQ ID NO: 182 - protein, 15 aa, Artificial Sequence
  SOURCE 1..15: /mol_type=“protein” /note=“shared tumour antigen” /organism=“Artificial Sequence”
CEFHACWPAFTVLGE

SEQ ID NO: 183 - protein, 10 aa, Artificial Sequence
  SOURCE 1..10: /mol_type=“protein” /note=“shared tumour antigen” /organism=“Artificial Sequence”
EVISCKLIKR

SEQ ID NO: 184 - protein, 17 aa, Artificial Sequence
  SOURCE 1..17: /mol_type=“protein” /note=“shared tumour antigen” /organism=“Artificial Sequence”
CATWKVICKSCISQTPG

SEQ ID NO: 185 - protein, 9 aa, Artificial Sequence
  SOURCE 1..9: /mol_type=“protein” /note=“tumour differentiation antigen” /organism=“Artificial Sequence”
YLSGANLNL

SEQ ID NO: 186 - protein, 9 aa, Artificial Sequence
  SOURCE 1..9: /mol_type=“protein” /note=“tumour differentiation antigen” /organism=“Artificial Sequence”
IMIGVLVGV

SEQ ID NO: 187 - protein, 9 aa, Artificial Sequence
  SOURCE 1..9: /mol_type=“protein” /note=“tumour differentiation antigen” /organism=“Artificial Sequence”
GVLVGVALI

SEQ ID NO: 188 - protein, 9 aa, Artificial Sequence
  SOURCE 1..9: /mol_type=“protein” /note=“tumour differentiation antigen” /organism=“Artificial Sequence”
HLFGYSWYK

SEQ ID NO: 189 - protein, 10 aa, Artificial Sequence
  SOURCE 1..10: /mol_type=“protein” /note=“tumour differentiation antigen” /organism=“Artificial Sequence”
QYSWFVNGTF

SEQ ID NO: 190 - protein, 9 aa, Artificial Sequence
  SOURCE 1..9: /mol_type=“protein” /note=“tumour differentiation antigen” /organism=“Artificial Sequence”
TYACFVSNL

SEQ ID NO: 191 - protein, 15 aa, Artificial Sequence
  SOURCE 1..15: /mol_type=“protein” /note=“tumour differentiation antigen” /organism=“Artificial Sequence”
AYVCGIQNSVSANRS

SEQ ID NO: 192 - protein, 25 aa, Artificial Sequence
  SOURCE 1..25: /mol_type=“protein” /note=“tumour differentiation antigen” /organism=“Artificial Sequence”
DTGFYTLHVIKSDLVNEEATGQFRV

SEQ ID NO: 193 - protein, 15 aa, Artificial Sequence
  SOURCE 1..15: /mol_type=“protein” /note=“tumour differentiation antigen” /organism=“Artificial Sequence”
YSWRINGIPQQHTQV

SEQ ID NO: 194 - protein, 13 aa, Artificial Sequence
  SOURCE 1..13: /mol_type=“protein” /note=“tumour differentiation antigen” /organism=“Artificial Sequence”
TYYRPGVNLSLSC

SEQ ID NO: 195 - protein, 13 aa, Artificial Sequence
  SOURCE 1..13: /mol_type=“protein” /note=“tumour differentiation antigen” /organism=“Artificial Sequence”
EIIYPNASLLIQN

SEQ ID NO: 196 - protein, 15 aa, Artificial Sequence
  SOURCE 1..15: /mol_type=“protein” /note=“tumour differentiation antigen” /organism=“Artificial Sequence”
YACFVSNLATGRNNS

SEQ ID NO: 197 - protein, 13 aa, Artificial Sequence
  SOURCE 1..13: /mol_type=“protein” /note=“tumour differentiation antigen” /organism=“Artificial Sequence”
LWWVNNQSLPVSP

SEQ ID NO: 198 - protein, 13 aa, Artificial Sequence
  SOURCE 1..13: /mol_type=“protein” /note=“tumour differentiation antigen” /organism=“Artificial Sequence”
LWWVNNQSLPVSP

SEQ ID NO: 199 - protein, 13 aa, Artificial Sequence
  SOURCE 1..13: /mol_type=“protein” /note=“tumour differentiation antigen” /organism=“Artificial Sequence”
LWWVNNQSLPVSP

SEQ ID NO: 200 - protein, 13 aa, Artificial Sequence
  SOURCE 1..13: /mol_type=“protein” /note=“tumour differentiation antigen” /organism=“Artificial Sequence”
EIIYPNASLLIQN

SEQ ID NO: 201 - protein, 13 aa, Artificial Sequence
  SOURCE 1..13: /mol_type=“protein” /note=“tumour differentiation antigen” /organism=“Artificial Sequence”
NSIVKSITVSASG

SEQ ID NO: 202 - protein, 9 aa, Artificial Sequence
  SOURCE 1..9: /mol_type=“protein” /note=“tumour differentiation antigen” /organism=“Artificial Sequence”
KTWGQYWQV

SEQ ID NO: 203 - protein, 10 aa, Artificial Sequence
  SOURCE 1..10: /mol_type=“protein” /note=“tumour differentiation antigen” /organism=“Artificial Sequence”
AMLGTHTMEV

SEQ ID NO: 204 - protein, 9 aa, Artificial Sequence
  SOURCE 1..9: /mol_type=“protein” /note=“tumour differentiation antigen” /organism=“Artificial Sequence”
ITDQVPFSV

SEQ ID NO: 205 - protein, 9 aa, Artificial Sequence
  SOURCE 1..9: /mol_type=“protein” /note=“tumour differentiation antigen” /organism=“Artificial Sequence”
YLEPGPVTA

SEQ ID NO: 206 - protein, 10 aa, Artificial Sequence
  SOURCE 1..10: /mol_type=“protein” /note=“tumour differentiation antigen” /organism=“Artificial Sequence”
LLDGTATLRL

SEQ ID NO: 207 - protein, 10 aa, Artificial Sequence
  SOURCE 1..10: /mol_type=“protein” /note=“tumour differentiation antigen” /organism=“Artificial Sequence”
VLYRYGSFSV

SEQ ID NO: 208 - protein, 10 aa, Artificial Sequence
  SOURCE 1..10: /mol_type=“protein” /note=“tumour differentiation antigen” /organism=“Artificial Sequence”
SLADTNSLAV

SEQ ID NO: 209 - protein, 9 aa, Artificial Sequence
  SOURCE 1..9: /mol_type=“protein” /note=“tumour differentiation antigen” /organism=“Artificial Sequence”
RLMKQDFSV

SEQ ID NO: 210 - protein, 9 aa, Artificial Sequence
  SOURCE 1..9: /mol_type=“protein” /note=“tumour differentiation antigen” /organism=“Artificial Sequence”
RLPRIFCSC

SEQ ID NO: 211 - protein, 9 aa, Artificial Sequence
  SOURCE 1..9: /mol_type=“protein” /note=“tumour differentiation antigen” /organism=“Artificial Sequence”
LIYRRRLMK

SEQ ID NO: 212 - protein, 9 aa, Artificial Sequence
  SOURCE 1..9: /mol_type=“protein” /note=“tumour differentiation antigen” /organism=“Artificial Sequence”
ALLAVGATK

SEQ ID NO: 213 - protein, 10 aa, Artificial Sequence
  SOURCE 1..10: /mol_type=“protein” /note=“tumour differentiation antigen” /organism=“Artificial Sequence”
IALNFPGSQK

SEQ ID NO: 214 - protein, 9 aa, Artificial Sequence
  SOURCE 1..9: /mol_type=“protein” /note=“tumour differentiation antigen” /organism=“Artificial Sequence”
ALNFPGSQK

SEQ ID NO: 215 - protein, 9 aa, Artificial Sequence
  SOURCE 1..9: /mol_type=“protein” /note=“tumour differentiation antigen” /organism=“Artificial Sequence”
ALNFPGSQK

SEQ ID NO: 216 - protein, 9 aa, Artificial Sequence
  SOURCE 1..9: /mol_type=“protein” /note=“tumour differentiation antigen” /organism=“Artificial Sequence”
VYFFLPDHL

SEQ ID NO: 217 - protein, 9 aa, Artificial Sequence
  SOURCE 1..9: /mol_type=“protein” /note=“tumour differentiation antigen” /organism=“Artificial Sequence”
RTKQLYPEW

SEQ ID NO: 218 - protein, 10 aa, Artificial Sequence
  SOURCE 1..10: /mol_type=“protein” /note=“tumour differentiation antigen” /organism=“Artificial Sequence”
HTMEVTVYHR

SEQ ID NO: 219 - protein, 9 aa, Artificial Sequence
  SOURCE 1..9: /mol_type=“protein” /note=“tumour differentiation antigen” /organism=“Artificial Sequence”
SSPGCQPPA

SEQ ID NO: 220 - protein, 10 aa, Artificial Sequence
  SOURCE 1..10: /mol_type=“protein” /note=“tumour differentiation antigen” /organism=“Artificial Sequence”
VPLDCVLYRY

SEQ ID NO: 221 - protein, 9 aa, Artificial Sequence
  SOURCE 1..9: /mol_type=“protein” /note=“tumour differentiation antigen” /organism=“Artificial Sequence”
LPHSSSHWL

SEQ ID NO: 222 - protein, 8 aa, Artificial Sequence
  SOURCE 1..8: /mol_type=“protein” /note=“tumour differentiation antigen” /organism=“Artificial Sequence”
SNDGPTLI

SEQ ID NO: 223 - protein, 15 aa, Artificial Sequence
  SOURCE 1..15: /mol_type=“protein” /note=“tumour differentiation antigen” /organism=“Artificial Sequence”
GRAMLGTHTMEVTVY

SEQ ID NO: 224 - protein, 16 aa, Artificial Sequence
  SOURCE 1..16: /mol_type=“protein” /note=“tumour differentiation antigen” /organism=“Artificial Sequence”
WNRQLYPEWTEAQRLD

SEQ ID NO: 225 - protein, 18 aa, Artificial Sequence
  SOURCE 1..18: /mol_type=“protein” /note=“tumour differentiation antigen” /organism=“Artificial Sequence”
TTEWVETTARELPIPEPE

SEQ ID NO: 226 - protein, 17 aa, Artificial Sequence
  SOURCE 1..17: /mol_type=“protein” /note=“tumour differentiation antigen” /organism=“Artificial Sequence”
TGRAMLGTHTMEVTVYH

SEQ ID NO: 227 - protein, 15 aa, Artificial Sequence
  SOURCE 1..15: /mol_type=“protein” /note=“tumour differentiation antigen” /organism=“Artificial Sequence”
GRAMLGTHTMEVTVY

SEQ ID NO: 228 - protein, 15 aa, Artificial Sequence
  SOURCE 1..15: /mol_type=“protein” /note=“tumour differentiation antigen” /organism=“Artificial Sequence”
SVSESDTIRSISIAS

SEQ ID NO: 229 - protein, 15 aa, Artificial Sequence
  SOURCE 1..15: /mol_type=“protein” /note=“tumour differentiation antigen” /organism=“Artificial Sequence”
LLANGRMPTVLQCVN

SEQ ID NO: 230 - protein, 15 aa, Artificial Sequence
  SOURCE 1..15: /mol_type=“protein” /note=“tumour differentiation antigen” /organism=“Artificial Sequence”
RMPTVLQCVNVSVVS

SEQ ID NO: 231 - protein, 9 aa, Artificial Sequence
  SOURCE 1..9: /mol_type=“protein” /note=“tumour differentiation antigen” /organism=“Artificial Sequence”
PLLENVISK

SEQ ID NO: 232 - protein, 10 aa, Artificial Sequence
  SOURCE 1..10: /mol_type=“protein” /note=“tumour differentiation antigen” /organism=“Artificial Sequence”
EAAGIGILTV

SEQ ID NO: 233 - protein, 9 aa, Artificial Sequence
  SOURCE 1..9: /mol_type=“protein” /note=“tumour differentiation antigen” /organism=“Artificial Sequence”
ILTVILGVL

SEQ ID NO: 234 - protein, 10 aa, Artificial Sequence
  SOURCE 1..10: /mol_type=“protein” /note=“tumour differentiation antigen” /organism=“Artificial Sequence”
EAAGIGILTV

SEQ ID NO: 235 - protein, 11 aa, Artificial Sequence
  SOURCE 1..11: /mol_type=“protein” /note=“tumour differentiation antigen” /organism=“Artificial Sequence”
AEEAAGIGILT

SEQ ID NO: 236 - protein, 11 aa, Artificial Sequence
  SOURCE 1..11: /mol_type=“protein” /note=“tumour differentiation antigen” /organism=“Artificial Sequence”
RNGYRALMDKS

SEQ ID NO: 237 - protein, 12 aa, Artificial Sequence
  SOURCE 1..12: /mol_type=“protein” /note=“tumour differentiation antigen” /organism=“Artificial Sequence”
EEAAGIGILTVI

SEQ ID NO: 238 - protein, 14 aa, Artificial Sequence
  SOURCE 1..14: /mol_type=“protein” /note=“tumour differentiation antigen” /organism=“Artificial Sequence”
AAGIGILTVILGVL

SEQ ID NO: 239 - protein, 13 aa, Artificial Sequence
  SOURCE 1..13: /mol_type=“protein” /note=“tumour differentiation antigen” /organism=“Artificial Sequence”
APPAYEKLPSAEQ

SEQ ID NO: 240 - protein, 12 aa, Artificial Sequence
  SOURCE 1..12: /mol_type=“protein” /note=“tumour differentiation antigen” /organism=“Artificial Sequence”
EEAAGIGILTVI

SEQ ID NO: 241 - protein, 23 aa, Artificial Sequence
  SOURCE 1..23: /mol_type=“protein” /note=“tumour differentiation antigen” /organism=“Artificial Sequence”
RNGYRALMDKSLHVGTQCALTRR

SEQ ID NO: 242 - protein, 20 aa, Artificial Sequence
  SOURCE 1..20: /mol_type=“protein” /note=“tumour differentiation antigen” /organism=“Artificial Sequence”
MPREDAHFIYGYPKKGHGHS

SEQ ID NO: 243 - protein, 20 aa, Artificial Sequence
  SOURCE 1..20: /mol_type=“protein” /note=“tumour differentiation antigen” /organism=“Artificial Sequence”
KNCEPVVPNAPPAYEKLSAE

SEQ ID NO: 244 - protein, 9 aa, Artificial Sequence
  SOURCE 1..9: /mol_type=“protein” /note=“tumour differentiation antigen” /organism=“Artificial Sequence”
SLSKILDTV

SEQ ID NO: 245 - protein, 9 aa, Artificial Sequence
  SOURCE 1..9: /mol_type=“protein” /note=“tumour differentiation antigen” /organism=“Artificial Sequence”
LYSACFWWL

SEQ ID NO: 246 - protein, 10 aa, Artificial Sequence
  SOURCE 1..10: /mol_type=“protein” /note=“tumour differentiation antigen” /organism=“Artificial Sequence”
FLTPKKLQCV

SEQ ID NO: 247 - protein, 10 aa, Artificial Sequence
  SOURCE 1..10: /mol_type=“protein” /note=“tumour differentiation antigen” /organism=“Artificial Sequence”
VISNDVCAQV

SEQ ID NO: 248 - protein, 9 aa, Artificial Sequence
  SOURCE 1..9: /mol_type=“protein” /note=“tumour differentiation antigen” /organism=“Artificial Sequence”
VLHWDPETV

SEQ ID NO: 249 - protein, 9 aa, Artificial Sequence
  SOURCE 1..9: /mol_type=“protein” /note=“tumour differentiation antigen” /organism=“Artificial Sequence”
MSLQRQFLR

SEQ ID NO: 250 - protein, 21 aa, Artificial Sequence
  SOURCE 1..21: /mol_type=“protein” /note=“tumour differentiation antigen” /organism=“Artificial Sequence”
ISPNSVFSQWRVVCDSLEDYD

SEQ ID NO: 251 - protein, 10 aa, Artificial Sequence
  SOURCE 1..10: /mol_type=“protein” /note=“tumour differentiation antigen” /organism=“Artificial Sequence”
SLPYWNFATG

SEQ ID NO: 252 - protein, 9 aa, Artificial Sequence
  SOURCE 1..9: /mol_type=“protein” /note=“tumour differentiation antigen” /organism=“Artificial Sequence”
SVYDFFVWL

SEQ ID NO: 253 - protein, 9 aa, Artificial Sequence
  SOURCE 1..9: /mol_type=“protein” /note=“tumour differentiation antigen” /organism=“Artificial Sequence”
TLDSQVMSL

SEQ ID NO: 254 - protein, 9 aa, Artificial Sequence
  SOURCE 1..9: /mol_type=“protein” /note=“tumour differentiation antigen” /organism=“Artificial Sequence”
LLGPGRPYR

SEQ ID NO: 255 - protein, 9 aa, Artificial Sequence
  SOURCE 1..9: /mol_type=“protein” /note=“tumour differentiation antigen” /organism=“Artificial Sequence”
LLGPGRPYR

SEQ ID NO: 256 - protein, 9 aa, Artificial Sequence
  SOURCE 1..9: /mol_type=“protein” /note=“tumour differentiation antigen” /organism=“Artificial Sequence”
ANDPIFVVL

SEQ ID NO: 257 - protein, 15 aa, Artificial Sequence
  SOURCE 1..15: /mol_type=“protein” /note=“tumour differentiation antigen” /organism=“Artificial Sequence”
QCTEVRADTRPWSGP

SEQ ID NO: 258 - protein, 10 aa, Artificial Sequence
  SOURCE 1..10: /mol_type=“protein” /note=“tumour differentiation antigen” /organism=“Artificial Sequence”
ALPYWNFATG

SEQ ID NO: 259 - protein, 9 aa, Artificial Sequence
  SOURCE 1..9: /mol_type=“protein” /note=“tumour differentiation antigen” /organism=“Artificial Sequence”
KCDICTDEY

SEQ ID NO: 260 - protein, 11 aa, Artificial Sequence
  SOURCE 1..11: /mol_type=“protein” /note=“tumour differentiation antigen” /organism=“Artificial Sequence”
SSDYVIPIGTY

SEQ ID NO: 261 - protein, 9 aa, Artificial Sequence
  SOURCE 1..9: /mol_type=“protein” /note=“tumour differentiation antigen” /organism=“Artificial Sequence”
MLLAVLYCL

SEQ ID NO: 262 - protein, 10 aa, Artificial Sequence
  SOURCE 1..10: /mol_type=“protein” /note=“tumour differentiation antigen” /organism=“Artificial Sequence”
CLLWSFQTSA

SEQ ID NO: 263 - protein, 9 aa, Artificial Sequence
  SOURCE 1..9: /mol_type=“protein” /note=“tumour differentiation antigen” /organism=“Artificial Sequence”
YMDGTMSQV

SEQ ID NO: 264 - protein, 9 aa, Artificial Sequence
  SOURCE 1..9: /mol_type=“protein” /note=“tumour differentiation antigen” /organism=“Artificial Sequence”
AFLPWHRLF

SEQ ID NO: 265 - protein, 9 aa, Artificial Sequence
  SOURCE 1..9: /mol_type=“protein” /note=“tumour differentiation antigen” /organism=“Artificial Sequence”
QCSGNFMGF

SEQ ID NO: 266 - protein, 12 aa, Artificial Sequence
  SOURCE 1..12: /mol_type=“protein” /note=“tumour differentiation antigen” /organism=“Artificial Sequence”
TPRLPSSADVEF

SEQ ID NO: 267 - protein, 9 aa, Artificial Sequence
  SOURCE 1..9: /mol_type=“protein” /note=“tumour differentiation antigen” /organism=“Artificial Sequence”
LPSSADVEF

SEQ ID NO: 268 - protein, 10 aa, Artificial Sequence
  SOURCE 1..10: /mol_type=“protein” /note=“tumour differentiation antigen” /organism=“Artificial Sequence”
LHHAFVDSIF

SEQ ID NO: 269 - protein, 9 aa, Artificial Sequence
  SOURCE 1..9: /mol_type=“protein” /note=“tumour differentiation antigen” /organism=“Artificial Sequence”
SEIWRDIDF

SEQ ID NO: 270 - protein, 15 aa, Artificial Sequence
  SOURCE 1..15: /mol_type=“protein” /note=“tumour differentiation antigen” /organism=“Artificial Sequence”
QNILLSNAPLGPQFP

SEQ ID NO: 271 - protein, 13 aa, Artificial Sequence
  SOURCE 1..13: /mol_type=“protein” /note=“tumour differentiation antigen” /organism=“Artificial Sequence”
SYLQDSDPDSFQD

SEQ ID NO: 272 - protein, 21 aa, Artificial Sequence [sequence omitted]

SEQ ID NO: 273 - protein, 9 aa, Artificial Sequence
  SOURCE 1..9: /mol_type=“protein” /note=“antigen overexpressed in tumour” /organism=“Artificial Sequence”
SVASTITGV

SEQ ID NO: 274 - protein, 10 aa, Artificial Sequence
  SOURCE 1..10: /mol_type=“protein” /note=“antigen overexpressed in tumour” /organism=“Artificial Sequence”
RSDSGQQARY

SEQ ID NO: 275 - protein, 9 aa, Artificial Sequence
  SOURCE 1..9: /mol_type=“protein” /note=“antigen overexpressed in tumour” /organism=“Artificial Sequence”
LLYKLADLI

SEQ ID NO: 276 - protein, 10 aa, Artificial Sequence
  SOURCE 1..10: /mol_type=“protein” /note=“antigen overexpressed in tumour” /organism=“Artificial Sequence”
YLNDHLEPWI

SEQ ID NO: 277 - protein, 9 aa, Artificial Sequence
  SOURCE 1..9: /mol_type=“protein” /note=“antigen overexpressed in tumour” /organism=“Artificial Sequence”
CQWGRLWQL

SEQ ID NO: 278 - protein, 10 aa, Artificial Sequence
  SOURCE 1..10: /mol_type=“protein” /note=“antigen overexpressed in tumour” /organism=“Artificial Sequence”
VLLQAGSLHA

SEQ ID NO: 279 - protein, 9 aa, Artificial Sequence
  SOURCE 1..9: /mol_type=“protein” /note=“antigen overexpressed in tumour” /organism=“Artificial Sequence”
KVHPVIWSL

SEQ ID NO: 280 - protein, 10 aa, Artificial Sequence
  SOURCE 1..10: /mol_type=“protein” /note=“antigen overexpressed in tumour” /organism=“Artificial Sequence”
LMLQNALTTM

SEQ ID NO: 281 - protein, 9 aa, Artificial Sequence
  SOURCE 1..9: /mol_type=“protein” /note=“antigen overexpressed in tumour” /organism=“Artificial Sequence”
LLGATCMFV

SEQ ID NO: 282 - protein, 15 aa, Artificial Sequence
  SOURCE 1..15: /mol_type=“protein” /note=“antigen overexpressed in tumour” /organism=“Artificial Sequence”
NPPSMVAAGSVVAAV

SEQ ID NO: 283 - protein, 10 aa, Artificial Sequence
  SOURCE 1..10: /mol_type=“protein” /note=“antigen overexpressed in tumour” /organism=“Artificial Sequence”
ALGGHPLLGV

SEQ ID NO: 284 - protein, 9 aa, Artificial Sequence
  SOURCE 1..9: /mol_type=“protein” /note=“antigen overexpressed in tumour” /organism=“Artificial Sequence”
TMNGSKSPV

SEQ ID NO: 285 - protein, 9 aa, Artificial Sequence
  SOURCE 1..9: /mol_type=“protein” /note=“antigen overexpressed in tumour” /organism=“Artificial Sequence”
RYQLDPKFI

SEQ ID NO: 286 - protein, 12 aa, Artificial Sequence
  SOURCE 1..12: /mol_type=“protein” /note=“antigen overexpressed in tumour” /organism=“Artificial Sequence”
DVTFNIICKKCG

SEQ ID NO: 287 - protein, 9 aa, Artificial Sequence
  SOURCE 1..9: /mol_type=“protein” /note=“antigen overexpressed in tumour” /organism=“Artificial Sequence”
FMVEDETVL

SEQ ID NO: 288 - protein, 10 aa, Artificial Sequence
  SOURCE 1..10: /mol_type=“protein” /note=“antigen overexpressed in tumour” /organism=“Artificial Sequence”
FINDEIFVEL

SEQ ID NO: 289 - protein, 9 aa, Artificial Sequence
  SOURCE 1..9: /mol_type=“protein” /note=“antigen overexpressed in tumour” /organism=“Artificial Sequence”
KYDCFLHPF

SEQ ID NO: 290 - protein, 9 aa, Artificial Sequence
  SOURCE 1..9: /mol_type=“protein” /note=“antigen overexpressed in tumour” /organism=“Artificial Sequence”
KYVGIEREM

SEQ ID NO: 291 - protein, 9 aa, Artificial Sequence
  SOURCE 1..9: /mol_type=“protein” /note=“antigen overexpressed in tumour” /organism=“Artificial Sequence”
NTYASPRFK

SEQ ID NO: 292 - protein, 9 aa, Artificial Sequence
  SOURCE 1..9: /mol_type=“protein” /note=“antigen overexpressed in tumour” /organism=“Artificial Sequence”
HLSTAFARV

SEQ ID NO: 293 - protein, 9 aa, Artificial Sequence
  SOURCE 1..9: /mol_type=“protein” /note=“antigen overexpressed in tumour” /organism=“Artificial Sequence”
KIFGSLAFL

SEQ ID NO: 294 - protein, 9 aa, Artificial Sequence
  SOURCE 1..9: /mol_type=“protein” /note=“antigen overexpressed in tumour” /organism=“Artificial Sequence”
IISAVVGIL

SEQ ID NO: 295 - protein, 9 aa, Artificial Sequence
  SOURCE 1..9: /mol_type=“protein” /note=“antigen overexpressed in tumour” /organism=“Artificial Sequence”
ALCRWGLLL

SEQ ID NO: 296 - protein, 9 aa, Artificial Sequence
  SOURCE 1..9: /mol_type=“protein” /note=“antigen overexpressed in tumour” /organism=“Artificial Sequence”
ILHNGAYSL

SEQ ID NO: 297 - protein, 9 aa, Artificial Sequence
  SOURCE 1..9: /mol_type=“protein” /note=“antigen overexpressed in tumour” /organism=“Artificial Sequence”
RLLQETELV

SEQ ID NO: 298 - protein, 9 aa, Artificial Sequence
  SOURCE 1..9: /mol_type=“protein” /note=“antigen overexpressed in tumour” /organism=“Artificial Sequence”
VVLGVVFGI

SEQ ID NO: 299 - protein, 10 aa, Artificial Sequence
  SOURCE 1..10: /mol_type=“protein” /note=“antigen overexpressed in tumour” /organism=“Artificial Sequence”
YMIMVKCWMI

SEQ ID NO: 300 - protein, 9 aa, Artificial Sequence
  SOURCE 1..9: /mol_type=“protein” /note=“antigen overexpressed in tumour” /organism=“Artificial Sequence”
HLYQGCQVV

SEQ ID NO: 301 - protein, 10 aa, Artificial Sequence
  SOURCE 1..10: /mol_type=“protein” /note=“antigen overexpressed in tumour” /organism=“Artificial Sequence”
YLVPQQGFFC

SEQ ID NO: 302 - protein, 9 aa, Artificial Sequence
  SOURCE 1..9: /mol_type=“protein” /note=“antigen overexpressed in tumour” /organism=“Artificial Sequence”
PLQPEQLQV

SEQ ID NO: 303 - protein, 9 aa, Artificial Sequence
  SOURCE 1..9: /mol_type=“protein” /note=“antigen overexpressed in tumour” /organism=“Artificial Sequence”
TLEEITGYL

SEQ ID NO: 304 - protein, 9 aa, Artificial Sequence
  SOURCE 1..9: /mol_type=“protein” /note=“antigen overexpressed in tumour” /organism=“Artificial Sequence”
ALIHHNTHL

SEQ ID NO: 305 - protein, 9 aa, Artificial Sequence
  SOURCE 1..9: /mol_type=“protein” /note=“antigen overexpressed in tumour” /organism=“Artificial Sequence”
PLTSIISAV

SEQ ID NO: 306 - protein, 9 aa, Artificial Sequence
  SOURCE 1..9: /mol_type=“protein” /note=“antigen overexpressed in tumour” /organism=“Artificial Sequence”
VLRENTSPK

SEQ ID NO: 307 - protein, 9 aa, Artificial Sequence
  SOURCE 1..9: /mol_type=“protein” /note=“antigen overexpressed in tumour” /organism=“Artificial Sequence”
TYLPTNASL

SEQ ID NO: 308 - protein, 9 aa, Artificial Sequence
  SOURCE 1..9: /mol_type=“protein” /note=“antigen overexpressed in tumour” /organism=“Artificial Sequence”
ALLEIASCL

SEQ ID NO: 309 - protein, 9 aa, Artificial Sequence
  SOURCE 1..9: /mol_type=“protein” /note=“antigen overexpressed in tumour” /organism=“Artificial Sequence”
WLPFGFILI

SEQ ID NO: 310 - protein, 9 aa, Artificial Sequence
  SOURCE 1..9: /mol_type=“protein” /note=“antigen overexpressed in tumour” /organism=“Artificial Sequence”
SPRWWPTCL

SEQ ID NO: 311 - protein, 9 aa, Artificial Sequence
  SOURCE 1..9: /mol_type=“protein” /note=“antigen overexpressed in tumour” /organism=“Artificial Sequence”
GVALQTMKQ

SEQ ID NO: 312 - protein, 9 aa, Artificial Sequence
  SOURCE 1..9: /mol_type=“protein” /note=“antigen overexpressed in tumour” /organism=“Artificial Sequence”
FMNKFIYEI

SEQ ID NO: 313 - protein, 10 aa, Artificial Sequence
  SOURCE 1..10: /mol_type=“protein” /note=“antigen overexpressed in tumour” /organism=“Artificial Sequence”
QLAVSVILRV

SEQ ID NO: 314 - protein, 14 aa, Artificial Sequence
  SOURCE 1..14: /mol_type=“protein” /note=“antigen overexpressed in tumour” /organism=“Artificial Sequence”
LPAVVGLSPGEQEY

SEQ ID NO: 315 - protein, 16 aa, Artificial Sequence
  SOURCE 1..16: /mol_type=“protein” /note=“antigen overexpressed in tumour” /organism=“Artificial Sequence”
VGQDVSVLFRVTGALQ

SEQ ID NO: 316 - protein, 8 aa, Artificial Sequence
  SOURCE 1..8: /mol_type=“protein” /note=“antigen overexpressed in tumour” /organism=“Artificial Sequence”
VLFYLGQY

SEQ ID NO: 317 - protein, 9 aa, Artificial Sequence
  SOURCE 1..9: /mol_type=“protein” /note=“antigen overexpressed in tumour” /organism=“Artificial Sequence”
TLNDECWPA

SEQ ID NO: 318 - protein, 9 aa, Artificial Sequence
  SOURCE 1..9: /mol_type=“protein” /note=“antigen overexpressed in tumour” /organism=“Artificial Sequence”
GLPPDVQRV

SEQ ID NO: 319 - protein, 12 aa, Artificial Sequence
  SOURCE 1..12: /mol_type=“protein” /note=“antigen overexpressed in tumour” /organism=“Artificial Sequence”
SLFPNSPKWTSK

SEQ ID NO: 320 - protein, 9 aa, Artificial Sequence
  SOURCE 1..9: /mol_type=“protein” /note=“antigen overexpressed in tumour” /organism=“Artificial Sequence”
STAPPVHNV

SEQ ID NO: 321 - protein, 9 aa, Artificial Sequence
  SOURCE 1..9: /mol_type=“protein” /note=“antigen overexpressed in tumour” /organism=“Artificial Sequence”
LLLLTVLTV

SEQ ID NO: 322 - protein, 12 aa, Artificial Sequence
  SOURCE 1..12: /mol_type=“protein” /note=“antigen overexpressed in tumour” /organism=“Artificial Sequence”
PGSTAPPAHGVT

SEQ ID NO: 323 - protein, 9 aa, Artificial Sequence
  SOURCE 1..9: /mol_type=“protein” /note=“antigen overexpressed in tumour” /organism=“Artificial Sequence”
LLGRNSFEV

SEQ ID NO: 324 - protein, 9 aa, Artificial Sequence
  SOURCE 1..9: /mol_type=“protein” /note=“antigen overexpressed in tumour” /organism=“Artificial Sequence”
RMPEAAPPV

SEQ ID NO: 325 - protein, 9 aa, Artificial Sequence
  SOURCE 1..9: /mol_type=“protein” /note=“antigen overexpressed in tumour” /organism=“Artificial Sequence”
SQKTYQGSY

SEQ ID NO: 326 - protein, 13 aa, Artificial Sequence
  SOURCE 1..13: /mol_type=“protein” /note=“antigen overexpressed in tumour” /organism=“Artificial Sequence”
PGTRVRAMAIYKQ

SEQ ID NO: 327 - protein, 12 aa, Artificial Sequence
  SOURCE 1..12: /mol_type=“protein” /note=“antigen overexpressed in tumour” /organism=“Artificial Sequence”
HLIRVEGNLRVE

SEQ ID NO: 328 - protein, 9 aa, Artificial Sequence
  SOURCE 1..9: /mol_type=“protein” /note=“antigen overexpressed in tumour” /organism=“Artificial Sequence”
TLPGYPPHV

SEQ ID NO: 329 - protein, 12 aa, Artificial Sequence
  SOURCE 1..12: /mol_type=“protein” /note=“antigen overexpressed in tumour” /organism=“Artificial Sequence”
CTACRWKKACQR

SEQ ID NO: 330 - protein, 9 aa, Artificial Sequence
  SOURCE 1..9: /mol_type=“protein” /note=“antigen overexpressed in tumour” /organism=“Artificial Sequence”
VLDGLDVLL

SEQ ID NO: 331 - protein, 10 aa, Artificial Sequence
  SOURCE 1..10: /mol_type=“protein” /note=“antigen overexpressed in tumour” /organism=“Artificial Sequence”
SLYSFPEPEA

SEQ ID NO: 332 - protein, 10 aa, Artificial Sequence
  SOURCE 1..10: /mol_type=“protein” /note=“antigen overexpressed in tumour” /organism=“Artificial Sequence”
ALYVDSLFFL

SEQ ID NO: 333 - protein, 9 aa, Artificial Sequence
  SOURCE 1..9: /mol_type=“protein” /note=“antigen overexpressed in tumour” /organism=“Artificial Sequence”
SLLQHLIGL

SEQ ID NO: 334 - protein, 9 aa, Artificial Sequence
  SOURCE 1..9: /mol_type=“protein” /note=“antigen overexpressed in tumour” /organism=“Artificial Sequence”
LYVDSLFFL

SEQ ID NO: 335 - protein, 9 aa, Artificial Sequence
  SOURCE 1..9: /mol_type=“protein” /note=“antigen overexpressed in tumour” /organism=“Artificial Sequence”
NYARTEDFF

SEQ ID NO: 336 - protein, 9 aa, Artificial Sequence
  SOURCE 1..9: /mol_type=“protein” /note=“antigen overexpressed in tumour” /organism=“Artificial Sequence”
LKLSGVVRL

SEQ ID NO: 337 - protein, 10 aa, Artificial Sequence
  SOURCE 1..10: /mol_type=“protein” /note=“antigen overexpressed in tumour” /organism=“Artificial Sequence”
PLPPARNGGL

SEQ ID NO: 338 - protein, 10 aa, Artificial Sequence
  SOURCE 1..10: /mol_type=“protein” /note=“antigen overexpressed in tumour” /organism=“Artificial Sequence”
SPSSNRIRNT

SEQ ID NO: 339 - protein, 9 aa, Artificial Sequence
  SOURCE 1..9: /mol_type=“protein” /note=“antigen overexpressed in tumour” /organism=“Artificial Sequence”
LAALPHSCL

SEQ ID NO: 340 - protein, 10 aa, Artificial Sequence
  SOURCE 1..10: /mol_type=“protein” /note=“antigen overexpressed in tumour” /organism=“Artificial Sequence”
GLASFKSFLK

SEQ ID NO: 341 - protein, 10 aa, Artificial Sequence
  SOURCE 1..10: /mol_type=“protein” /note=“antigen overexpressed in tumour” /organism=“Artificial Sequence”
RAGLQVRKNK

SEQ ID NO: 342 - protein, 10 aa, Artificial Sequence
  SOURCE 1..10: /mol_type=“protein” /note=“antigen overexpressed in tumour” /organism=“Artificial Sequence”
ALWPWLLMAT

SEQ ID NO: 343 - protein, 9 aa, Artificial Sequence
  SOURCE 1..9: /mol_type=“protein” /note=“antigen overexpressed in tumour” /organism=“Artificial Sequence”
NSQPVWLCL

SEQ ID NO: 344 - protein, 9 aa, Artificial Sequence
  SOURCE 1..9: /mol_type=“protein” /note=“antigen overexpressed in tumour” /organism=“Artificial Sequence”
LPRWPPPQL

SEQ ID NO: 345 - protein, 9 aa, Artificial Sequence
  SOURCE 1..9: /mol_type=“protein” /note=“antigen overexpressed in tumour” /organism=“Artificial Sequence”
KMDAEHPEL

SEQ ID NO: 346 - protein, 9 aa, Artificial Sequence
  SOURCE 1..9: /mol_type=“protein” /note=“antigen overexpressed in tumour” /organism=“Artificial Sequence”
AWISKPPGV

SEQ ID NO: 347 - protein, 10 aa, Artificial Sequence
  SOURCE 1..10: /mol_type=“protein” /note=“antigen overexpressed in tumour” /organism=“Artificial Sequence”
SAWISKPPGV

SEQ ID NO: 348 - protein, 9 aa, Artificial Sequence
  SOURCE 1..9: /mol_type=“protein” /note=“antigen overexpressed in tumour” /organism=“Artificial Sequence”
MIAVFLPIV

SEQ ID NO: 349 - protein, 15 aa, Artificial Sequence
  SOURCE 1..15: /mol_type=“protein” /note=“antigen overexpressed in tumour” /organism=“Artificial Sequence”
HQQYFYKIPILVINK

SEQ ID NO: 350 - protein, 10 aa, Artificial Sequence
  SOURCE 1..10: /mol_type=“protein” /note=“antigen overexpressed in tumour” /organism=“Artificial Sequence”
ELTLGEFLKL

SEQ ID NO: 351 - protein, 9 aa, Artificial Sequence
  SOURCE 1..9: /mol_type=“protein” /note=“antigen overexpressed in tumour” /organism=“Artificial Sequence”
ILAKFLHWL

SEQ ID NO: 352 - protein, 9 aa, Artificial Sequence
  SOURCE 1..9: /mol_type=“protein” /note=“antigen overexpressed in tumour” /organism=“Artificial Sequence”
RLVDDFLLV

SEQ ID NO: 353 - protein, 15 aa, Artificial Sequence
  SOURCE 1..15: /mol_type=“protein” /note=“antigen overexpressed in tumour” /organism=“Artificial Sequence”
RPGLLGASVLGLDDI

SEQ ID NO: 354 - protein, 15 aa, Artificial Sequence
  SOURCE 1..15: /mol_type=“protein” /note=“antigen overexpressed in tumour” /organism=“Artificial Sequence”
LTDLQPYMRQFVAHL

SEQ ID NO: 355 - protein, 9 aa, Artificial Sequence
  SOURCE 1..9: /mol_type=“protein” /note=“antigen overexpressed in tumour” /organism=“Artificial Sequence”
SRFGGAVVR

SEQ ID NO: 356 - protein, 11 aa, Artificial Sequence
  SOURCE 1..11: /mol_type=“protein” /note=“antigen overexpressed in tumour” /organism=“Artificial Sequence”
TSEKRPFMCAY

SEQ ID NO: 357 - protein, 9 aa, Artificial Sequence
  SOURCE 1..9: /mol_type=“protein” /note=“antigen overexpressed in tumour” /organism=“Artificial Sequence”
CMTWNQMNL

SEQ ID NO: 358 - protein, 11 aa, Artificial Sequence
  SOURCE 1..11: /mol_type=“protein” /note=“antigen overexpressed in tumour” /organism=“Artificial Sequence”
LSHLQMHSRKH

SEQ ID NO: 359 - protein, 16 aa, Artificial Sequence
  SOURCE 1..16: /mol_type=“protein” /note=“antigen overexpressed in tumour” /organism=“Artificial Sequence”
KRYFKLSHLQMHSRKH